(19) United States
(12) Patent Application Publication
(10) Pub. No.: US 2025/0352506 A1
(43) Pub. Date: Nov. 20, 2025

(54) MRGPRB2/X2, AGONIST AND ANTAGONIST AND METHODS THEREOF

(71) Applicant: THE UNIVERSITY OF HONG KONG, Hong Kong (CN)

(72) Inventors: Karthi Duraisamy, Hong Kong (CN); Billy Kwok Chong Chow, Hong Kong (CN)

(73) Assignee: THE UNIVERSITY OF HONG KONG, Hong Kong (CN)

(21) Appl. No.: 18/667,291

(22) Filed: May 17, 2024

(51) Int. Cl. A61K 31/24; A61P 1/00; A61P 29/00

(52) U.S. Cl. CPC A61K 31/24; A61P 1/00; A61P 29/00

(57) ABSTRACT

The present disclosure is directed to use of MrgprX2 agonist and antagonists in the treatment of inflammatory disorders, e.g., inflammatory disorders of the digestive tract. This disclosure is also directed to pharmaceutical compositions comprising a MrgprX2 agonist, antagonist and a pharmaceutically or orally acceptable carrier for administration. In one embodiment, the MrgprX2 antagonist having the formula:

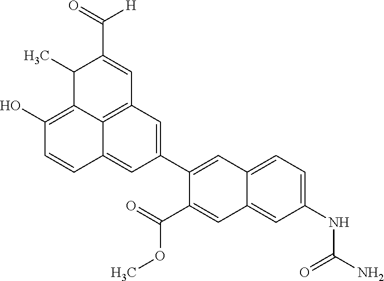 [SMILES: COC(=O)c1cc2cc(NC(N)=O)ccc2cc1-c1cc2c3c(c(O)ccc3c1)C(C)C(C=O)=C2]

### 1. FIELD

The disclosure is generally directed to a MRGPRB2/X2, its agonist, antagonist and methods of treatment and preparation.

### SEQUENCE LISTING

The instant application contains a Sequence Listing which has been submitted electronically in XML file format and is hereby incorporated by reference in its entirety. Said XML copy, created on May ______, 2024, is named 10030_012538-US0_SLxml and is ______ bytes in size.

### 2. BACKGROUND

Ulcerative colitis (UC) is a chronic inflammatory and a potentially life-threatening condition of the intestinal tract [1]. UC and Crohn's disease are chronic and recurrent inflammatory bowel diseases (IBD) that severely affect the gastrointestinal tract [2]. The precise etiology is still a mystery and the cure for the disease is unlikely [3]. The interaction between multiple factors such as genetic, environmental, gut microbiome and immune system influences the dysregulated immune response to induce UC [4, 5]. Major symptoms of UC include diarrhoea, abdominal pain, rectal bleeding, and weight loss [1]. There are multiple murine models developed to study colitis but chemically induced colitis model by either dextran sulfate sodium (DSS) and 2,4,6-trinitrobenzenesulfonic acid are preferred due to their ease of use, onset, duration, and controlled colitis severity [6]. Nowadays, DSS-induced colitis mice model has been the prevalently used model for colitis, and severe colitis can be induced with 40-50 kDa DSS in drinking water [6, 7]. Even though, colitis etiology is complex and multiple factors are known to be involved, there are several GPCRs implicated in the development and pathology of colitis [8, 9]. Consequently, GPCRs are the major target for drug development for various IBD diseases including colitis [10]since they are the largest group of cell surface receptors found in humans that regulate a plethora of cellular responses [111]. Out of an estimated 367 endoGPCRs in the human genome, 124 are listed in FDA's orange book as the drug targets for various diseases. Indeed, almost half of all currently available drugs on the market target GPCRs [12, 13], including some of the best-selling drugs such as Zantac (Ulcers) [14], Zyrtec (Allergy)[15], and Singulair (Asthma) [16].

Mas related G protein receptor X2 (Mrgprx2) and mast cells (MCs) have major roles in host defense, allergy, inflammation and immune regulation [17]. They are prevalently found in tissues that are exposed to the environment such as skin, airways, and gastrointestinal tract and act as the first line of defense against endogenous and environmental invaders [18]. They modulate the human immune system via degranulation of inflammatory mediators, lipid mediators, cytokines, histamines, and proteases, which are involved in innate and adaptive immunity [19]. Consequently, MC dysfunction leads to chronic allergic and inflammatory disorders, cancers and autoimmune diseases [20]. Interestingly, MC degranulation is stimulated by two different pathways depending on the protein being activated: IgE-dependent (FcεRI receptor) or IgE-independent (Mrgprx2) pathways [21, 22]. Activation of MC with Mrgprx2 agonists have been shown to induce neutrophil and monocyte recruitment [22, 23]. Several host defense peptides (HDPs) or anti-microbial peptides (AMPs) and small molecules have also been reported to act as agonists of Mrgprx2, including P17, cortistatin-14 (CST-14), protegrin, LL-37, substance P, PAMP, mastoparan, and compound 48/80 [22, 24] and other compounds like GE1111, quercetin and QWF have been classified as antagonists of Mrgprx2, at least in certain situations [25, 26]. In murine models, Mrgprb2 and Mrgpra1 are characterized as the mouse orthologue of human Mrgprx2 [27, 28]. There are substantial evidence that Mrgprx2 and MCs play a role in colitis [10, 29]. Higher levels of endogenous Mrgprx2 agonists such as PAMP-12 and LL-37 were observed in inflamed UC than uninflamed UC [10, 19, 30]. Mrgprb2 knockout mice has been recently used to demonstrate the role of Mrgprb2/x2 in colitis model [31]. However, there is very limited in vivo mechanistic studies on the role of Mrgprx2 in colitis [10]. Conflicting evidence on their involvement in pathogenesis and progression of UC open up several research questions. For instance, Mrgprx2 is often considered as the allergic receptor since its activation leads to various inflammatory immune cell recruitments, itch and pain [22, 23, 32], whereas Mrgprb2 knockout mice model aggravated the colitis instead of alleviating [31], which are often due to the inflammation caused by immune cell infiltration [33]. Therefore, it is important to provide agonists, antagonists of Mrgprb2/x2 for the treatment of UC.

### 3. SUMMARY

This disclosure is based, in part, on the identification of MRGPRX2 or MRGPRX2 ortholog modulator compounds. Among rodent orthologs, mouse mrgprb2 and rat mrgprb3 correspond functionally to human MRGPRX2 in mast cells. MRGPRX2 and its ortholog receptors mediate disorders including pseudo-allergic drug reactions, chronic itch (e.g., pruritus), inflammation disorders, pain disorders, a cancer associated condition, skin disorders, wound healing, cardiovascular disease, and lung inflammation/COPD. In one embodiment, expression of MRGPRX2 and its orthologs is largely restricted to mast cells. Mast cells are innate immune cells that primarily reside at sites exposed to the external environment, such as the skin, oral/gastrointestinal mucosa and respiratory tract. Mast cells express numerous receptors that respond to mechanical and chemical stimuli. Upon activation, classically by IgE, mast cells release pre-formed mediators from granules (e.g., histamine, proteases, and heparin) and newly synthesized mediators (e.g., thromboxane, prostaglandin D2, leukotriene C4, tumor necrosis factor alpha, eosinol chemotactor factor, and platelet-activating factor) that elicit allergic and inflammatory responses. Histamine dilates post-capillary venules, activates the endothelium, and increases blood vessel permeability. This causes local edema, warmth, redness, and chemotaxis of other inflammatory cells to the site of release. Histamine also contributes to neuronal sensitization that leads to pain or itch. MRGPRX2 and its orthologs mediate immunoglobulin E (IgE) independent activation of mast cells. MRGPRX2 and its orthologs are receptors for (or sensitive to activation by) various ligands, including basic secretagogues (small cationic molecules), certain drugs (e.g., cationic peptidergic drugs), neuropeptides, and antimicrobial peptides, and thus are important for non-IgE mediated pseudo-allergic reactions, inflammation, pain, and itch conditions. Mast cells may also contribute to the progression of autoimmune disorders by promoting chronic inflammation in the local tissue microenvironment and ultimately polarizing toward an immune response. Thus, modulating MRGPRX2 or MRGPRX2 orthologs allows for treatment of autoimmune diseases, pseudo-allergic drug reactions, pain, itch, and inflammatory disorders such as inflammatory bowel disease, urticaria, sinusitis, asthma, rosacea, endometriosis, and other MRGPRX2 or MRGPRX2 ortholog dependent conditions as explained in more detail below.

In one embodiment is provided a method of treating a MRGPRX2 or a MRGPRX2 ortholog dependent condition by administering to a subject in need thereof an effective amount of the pharmaceutical composition of the modulator compounds of the present invention.

Methods are provided for modulating MRGPRX2 generally, or for treating a MRGPRX2 or a MRGPRX2 ortholog dependent condition, more specifically, by contacting the MRGPRX2 or the MRGPRX2 ortholog by administering to a subject in need thereof, respectively, an effective amount of a compound having structure (I):

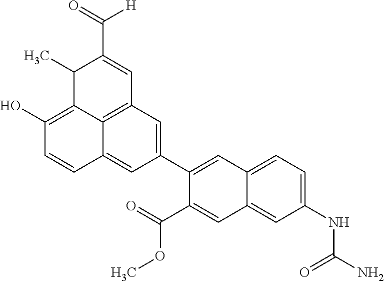 [SMILES: COC(=O)c1cc2cc(NC(N)=O)ccc2cc1-c1cc2c3c(c(O)ccc3c1)C(C)C(C=O)=C2]

or a pharmaceutically acceptable salt, isomer, hydrate, solvate or isotope thereof.

In another embodiment, methods are provided for treating an MRGPRX2 or a MRGPRX2 ortholog dependent condition by administering to a subject in need thereof an effective amount of a compound having structure (I), or a pharmaceutically acceptable salt, isomer, hydrate, solvate or isotope thereof.

In certain embodiments, the MRGPRX2 or a MRGPRX2 ortholog dependent condition is one or more of a pseudo-allergic reaction, itch associated condition, a pain associated condition, a cancer associated condition, an inflammation-associated condition, or an autoimmune disorder.

In one embodiment, the methods of treating the MRGPRX2 or the MRGPRX2 ortholog dependent condition are provided which comprises administering an effective amount of a compound of structure (I) as defined herein, or a pharmaceutically acceptable salt, isomer, hydrate, solvate or isotope thereof.

Provided is a method of treating a type 2 inflammation or mast cell-dependent disease in a subject in need thereof, comprising administering to the subject a therapeutically effective amount of the pharmaceutical composition disclosed herein.

Provided is a method of modulating a Mas-Related G-Protein Receptor (MRGPR) X2 or a MRGPRX2 ortholog by contacting MRGPRX2 or MRGPRX2 ortholog with an effective amount of the pharmaceutical composition disclosed herein.

Provided is a method of treating a MRGPRX2 or a MRGPRX2 ortholog dependent condition by administering to a subject in need thereof an effective amount of the pharmaceutical composition disclosed herein.

Provided is a method of treating a pseudo-allergic reaction, an itch associated condition, a pain associated condition, a cancer associated condition, an inflammatory or autoimmune disorder by administering to a subject in need thereof an effective amount of the pharmaceutical composition disclosed herein.

In one embodiment, the inflammatory disorder activates or is consequent to activation, of MrgprX2.

Provided is a method of treating a type 2 inflammation or a mast cell-dependent disease in a subject in need thereof, comprising administering to the subject a therapeutically effective amount of an MRGPRX2 binding molecule disclosed herein.

In one embodiment, the MRGPRX2 binding molecule is a MRGPRX2 inhibitor.

Provided is a method of treating a type 2 inflammation or mast cell-dependent disease in a subject in need thereof, comprising administering to the subject a therapeutically effective amount of a therapeutic composition comprising a MRGPRX2 binding molecule disclosed herein.

Provided is compound having formula I, or a pharmaceutically acceptable salt, hydrate, solvate or isotope thereof:

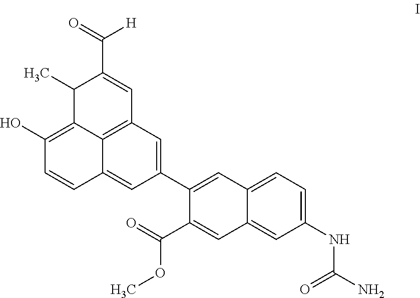 [SMILES: COC(=O)c1cc2cc(NC(N)=O)ccc2cc1-c1cc2c3c(c(O)ccc3c1)C(C)C(C=O)=C2]

Pharmaceutical compositions containing such compounds, as well as the compounds themselves, are also provided.

Provided herein are binding molecule of Mrgprb2/x2, including but not limited to agonists, antagonists of Mrgprb2/x2. In certain embodiments, provided are agonists P17, bioactive Ala-substituted P17 analogues (P17*A4 and P17*A7), and cortistatin 14 (“CST-14”), and the antagonist GE1111.

Provided herein is a treatment of ulcerative colitis (“UC”).

Provided herein is a mouse model of dextran sulfate sodium (“DSS”)-induced colitis.

Provided herein is a method for treating an inflammatory disorder, the method comprising administering to a subject in need thereof a composition comprising a therapeutically effective amount of compound having the structure:

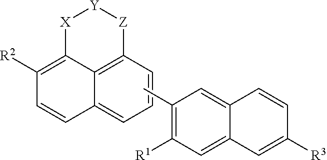

wherein each of R¹ and R³ is independently

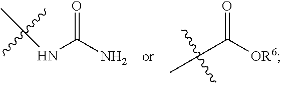

R² is H or OH;

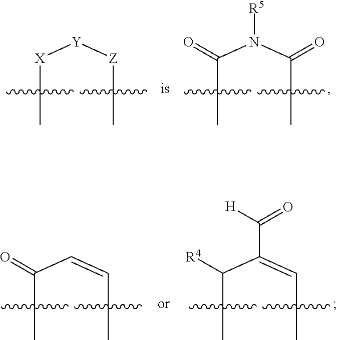

and
each of R⁴, R⁵ and R⁶ is independently H or alkyl; and a pharmaceutically acceptable carrier.

In one embodiment, the compound is represented by the structure:

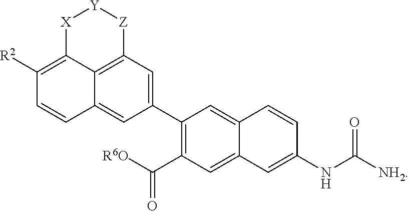

In one embodiment, the compound is represented by the structure:

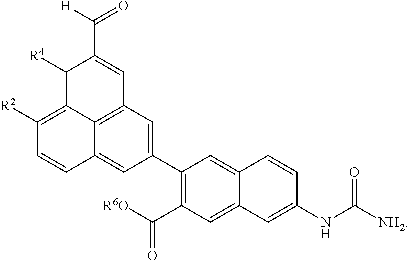

In one embodiment, the compound is represented by the structure:

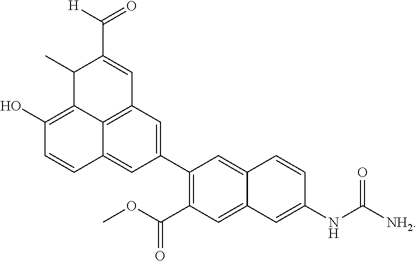 [SMILES: COC(=O)c1cc2cc(NC(N)=O)ccc2cc1-c1cc2c3c(c(O)ccc3c1)C(C)C(C=O)=C2]

In one embodiment, the compound is represented by the structure:

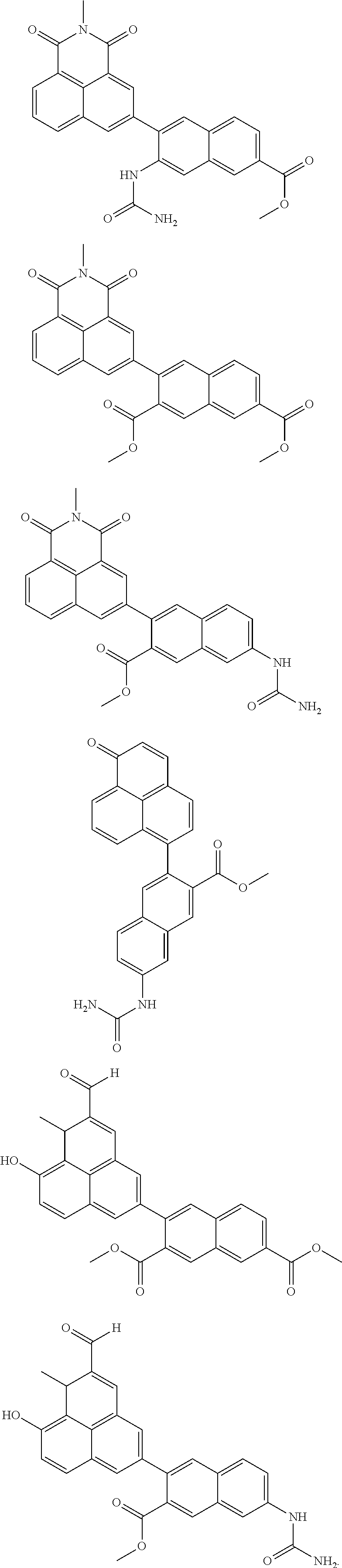

In one embodiment, the inflammatory disorder is an inflammation of the gastrointestinal tract. In one embodiment, the inflammatory disorder is inflammatory bowel disease (“IBD”), Crohn's disease (“CD”) or ulcerative colitis (“UC”).

In one embodiment, the composition is in the form of a cream, a gel, a spray, an ointment, or is a unit dosage form for oral administration.

In one embodiment, the compound is present at a concentration of about 0.001 wt. % to about 10 wt. %, based on the total weight of the composition.

In one embodiment, the compound is present at a concentration of about 0.1 wt. % to about 5 wt. %, based on the total weight of the composition.

In one embodiment, the subject is a mammal.

In one embodiment, the mammal is a human.

Provided is a pharmaceutical composition comprising the compound represented by the structure:

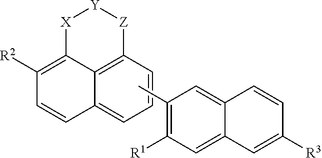

wherein each of R¹ and R³ is independently

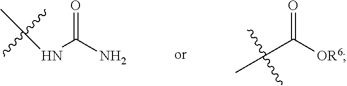

R² is H or OH;

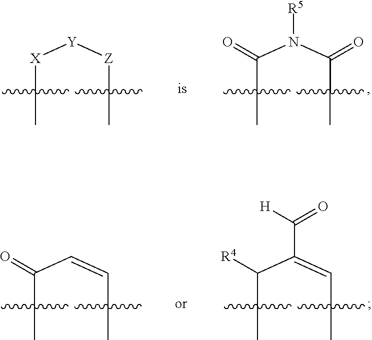

and
each of R⁴, R⁵ and R⁶ is independently H or alkyl, or a pharmaceutically acceptable salt, hydrate, solvate or isotope thereof and at least one pharmaceutically acceptable carrier.

In one embodiment, the compound is represented by the structure:

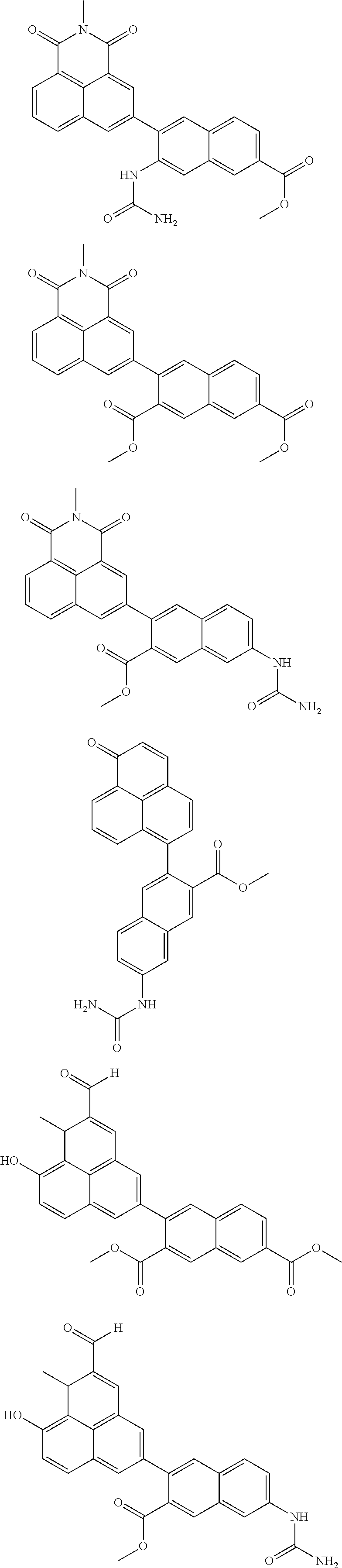

or a pharmaceutically acceptable salt, hydrate, solvate or isotope thereof and at least one pharmaceutically acceptable carrier.

### 4. BRIEF DESCRIPTION OF THE FIGURES

The patent or application file contains at least one drawing executed in color. Copies of this patent or patent application publication with color drawings will be provided by the Office upon request and payment of the necessary fee.

FIG. 1 Mrgprb2 knockout validation in Mrgprb2^{-/-} animals. Genotyping results shows successful knockout of Mrgprb2 in some animals. PCR was performed as instructed by The Centre for Phenogenomics. No bands in PCR set 1 shows KO mice. Only the smaller band in PCR set 2 represents the KO mice. Only verified mice were used in the experiments.

FIGS. 2A-G. Role of Mrgprb2 on the clinical parameters of DSS-induced colitis. C57BL/6 mice (WT) and Mrgprb2^{-/-} mice were supplied to with water containing 2% DSS (mixed in drinking water) from Day 0 to Day 5 and DSS solution was replaced with normal water cycle (ad libitum) from Day 5 to Day 12. The body weight of the mice was recorded daily. (A) Weight changes were calculated as a percentage of weight prior to 2% DSS treatment (Day 0). (B) the DAI was calculated by weight loss, stool consistency and rectal bleeding scoring (Table 1). (C) A representative photograph of colon tissues in each group is provided. (D) Colon length measured at Day 12. (E) Representative microscopic image of H&E-stained colon tissues of the experimental groups (4× magnification). (F) qRT-PCR analysis of colon integrity genes (E-cadherin, occludin) and neoangiogenic gene VEGF. (G) Representative IHC images of claudin-1 expression in colon. (N=5-6 per group). All data are expressed as means±SEM. *p<0.05, ** p<0.01 ***p<0.001 and****p<0.0001; ^{#}p<0.05, ^{##}p<001, ^{###}p<0.001 and ^{####}p<0.0001.

FIGS. 3A-B Role of Mrgprb2 on the clinical parameters of DSS-induced colitis. C57BL/6 mice (WT) and Mrgprb2^{-/-} mice were subjected to 2% DSS (mixed in drinking water) and water (without DSS) for Day 0 to Day 5 and DSS was replaced with normal water cycle (ad libitum) from Day 5 to Day 12. (Ai-ii) Occult blood with the score for blood stool, and (B) feces score. All data are expressed as means±SEM. *p<0.05, **p<0.01 ***p<0.001 and ****p<0.0001.

FIGS. 4A-C. Effect of Mrgprb2 gene knockout in immune cell infiltration in colon through altered cytokine release. (A) (i) Membrane arrays from WT and Mrgprb2^{-/-} animals treated with or without 2% DSS (mixed with drinking water). Positive control (dark blue boxes), blanks (orange boxes), and negative control (yellow boxes). Representative coloured boxes indicate the location of the detection of cytokines, M-CSF (red), LIX (blue), sTNF RI (green), TCA-3 (black) (ii) Graph indicating the overall fold change in cytokine release between treatment groups (N=2; data are pooled from 2 different individual experiments). (B) Representative graphs showing (i) M-CSF and (ii) LIX released in blood in different treatment groups. (C) Representative IHC images showing F4/80, CD38 and COX-2 markers in colon (N=5-6 per group). All data are expressed as means±SD. *p<0.05, **p<0.01, ***p<0.001 and ****p<0.0001; ^{#}p<0.05, ^{##}p<0.01, ^{###}p<0.001 and ^{####}p<0.0001.

FIGS. 5A-C Representative images of immune cell markers F4/80 (A) and CD68 (B) and COX-2(C) were shown in colonic epithelium of different treatment groups subjected to 2% DSS. Images were analyzed with ImageJ software by Image→Color→Colour Deconvolution method to split the images.

FIG. 6 Protective effects of Mrgprb2/x2 ligands on survival in a DSS-induced colitis model. (Ai) Kaplan-Meier survival curve of control and treatment group in 4% DSS-induced colitis (GraphPad Prism 8). A log-rank test verified significant differences between control treated group with P17, GE1111 and CST-14 treated mice groups (p-value<0.0001, 0.0002 and 0.0009 respectively). (N=10) (Aii) Severe symptoms of DSS induced colitis. (rectal bleeding and/or bloody diarrhoea—animals were sacrificed if they reach this condition).

FIGS. 7A-C. Effect of Mrgprb2/x2 ligands on the clinical parameters of DSS-induced colitis. C57BL/6 mice were subjected to 2.5% DSS (mixed in drinking water) from Day 0 to Day 5 and DSS solution was replaced with normal water cycle (ad libitum) from Day 5 to Day 12. Mice received no treatment (water+saline), P17 and P17 analogues, CST-14 and GE1111 every alternative days from Day 0 to Day12 (N=8 mice/group). The body weight of the mice were daily recorded. (A) (i) Weight changes were calculated as a percentage of weight prior to 2.5% DSS treatment (Day 0). (ii) the DAI was calculated by weight loss, stool consistency and rectal bleeding scoring (Table 1). (B) (i) A representative photograph of colon tissues in each group is provided. (ii) Colon length measured at Day 12. (iii) Colon weight/length ratio of mice indicating the severity of the colitis. (iv) FITC-dextran release in blood plasma (C) Representative microscopic image of H&E-stained colon tissues in each group (4× magnification) (N=8 per group). All data are expressed as means±SEM. *p<0.05, **p<0.01 ***p<0.001 and ****p<0.0001.

FIGS. 8A-C Role of Mrgprb2 on the clinical parameters of DSS-induced colitis. C57BL/6 mice (WT) were subjected to 2.5% DSS (mixed in drinking water) and water (without DSS) for Day 0 to Day 5 and DSS solution was replaced with normal water cycle (ad libitum) from Day 5 to Day 12. P17, P17 analogues, CST-14 and GE1111 were i.p. injected every alternative days from Day 0 to Day 12. (A) Occult blood (B) Feces score and (C) Colon weight. All data are expressed as means±SEM. *p<0.05, **p<0.01 ***p<0.001 and ****p<0.0001.

FIGS. 9A-E. Effects of Mrgprb2/x2 ligands on Mrgprx2 activation, internalisation and expression. (A) Dose response curve of Mrgprx2 ligands treatment showing the β-arrestin recruitment in Mrgprx2-transfected HTLA cells pre-treated with GE1111. (i) Compound 48/80, (ii) P17, (iii) P17*A4 and (iv) P17*A7. (v) Bar chart showing the dose response effect of GEl111 in Mrgprx2-transfected HTLA cells. Data are shown as means±SEM. (B) Representative western blot images showing the effect of Mrgprx2 ligands in LAD2 mast cells. (i) ERK1/2 (p-ERK1/2), STIM1 and GAPDH protein expression. Bar graphs showing the relative intensities of the bands for (ii) pERK1/2 and (iii) STIM1. Phosphorylated levels of protein were normalized to the total level of the respective proteins. Data are shown as means±SEM. (C) Flow cytometry analysis showing the surface and total expression of Mrgprx2 upon Mrgprb2/x2 ligand treatment (Compound 48/80, P17, P17*A4 and P17*A7-10 μM and GE1111 100 μM) after 1 hr. (D) Representative images of in situ hybridization analysis of Mrgprb2 expression in water and 2.5% DSS-treated mice and graph showing the percentage area intensity of Mrgprb2 expression in colon tissues (N=3). Data are expressed as means±SD (E) qRT-PCR expression of (i) Mrgpra1 and (ii) Mrgprb2 expression in animals treated with Mrgprb2/x2 ligands (N=6-8 and n=3). All data are expressed as means±SEM. *p<0.05, ** p<0.01 and ***p<0.001.

FIGS. 10A-B. Gene expression of colon integrity genes assessed by qRT-PCR and IHC analysis. (A) Representative graphs showing (i) claudin 1, (ii) occludin and (iii) E-cadherin and (iv) VEGF mRNA expression. (B) Representative IHC images of the expression of claudin 1 in the colonic epithelium and graph showing the percentage intensity of claudin 1 in different treatment groups (N=4 and n=3). All data are expressed as means±SEM. *p<0.05, **p<0.01, ***p<0.001 and ****p<0.0001.

FIG. 11 (A) Representative IF images of rabbit anti-claudin 1 antibody and Alexa-Fluor 488 donkey anti-rabbit secondary antibody in colonic epithelium of different treatment groups subjected to 2.5% DSS. Colon samples were counter stained with DAPI (blue signal).

FIGS. 12A-C. Effect of Mrgprb2/x2 ligands on the cytokine releases in the blood stream of the DSS-induced colitis animals using a mouse cytokine array and IHC analysis of colon. (A) (i) Membrane arrays with saline (without DSS), saline (with 2.5% DSS), P17*A7 (with 2.5% DSS) and GE1111 (with 2.5% DSS) treatment. Positive control (dark blue boxes), blanks (orange boxes) and negative control (yellow boxes). Representative coloured boxes indicate the location of the detection of cytokines, M-CSF (red), BLC (purple), sTNF RI (green), sTNF RII (black), G-CSF (blue), TCA-3 (white) and IL-1α (cyan) (N=2; data are pooled from 2 different individual experiments). (ii) Graph indicating the overall fold change in reactivity of cytokine release between treatment groups (Saline+Water, Saline+DSS, P17*A7+DSS and GE1111+DSS). (B) Representative graphs showing M-CSF, BLC, G-CSF and sTNF RI released in blood in different treatment groups. (C) IHC analysis of immune cell infiltration in the colon of DSS-induced colitis animals. Representative images of immune cell markers F4/80 (i), CD68 (ii) and, inflammatory marker COX-2 (iii) were shown in colonic epithelium of different treatment groups subjected to 2.5% DSS and graphs showing the immune cell infiltration and inflammation amount in the colons (N=4 and n=3). All data are expressed as means±SD. *p<0.05 ** p<0.01 ***p<0.001 and ****p<0.0001.

FIG. 13 (A) Representative images of immune cell marker CD68 were shown in colonic epithelium of different treatment groups subjected to 2.5% DSS. Images were analyzed with ImageJ software by Image→Color→Colour Deconvolution method to split the images.

FIG. 14 (A) Representative images of immune cell marker F4/80 were shown in colonic epithelium of different treatment groups subjected to 2.5% DSS. Images were analyzed with ImageJ software by Image→Color→Colour Deconvolution method to split the images.

FIG. 15 (A) Representative images of immune cell marker COX-2 were shown in colonic epithelium of different treatment groups subjected to 2.5% DSS. Images were analyzed with ImageJ software by Image→Color→Colour Deconvolution method to split the images.

FIGS. 16A-C Effects of Mrgprx2 ligands on mice. C57BL/6 mice (WT) were subjected to water (without DSS) from Day 1 to Day 12. P17, P17 analogues, and GE1111 were i.p. injected every alternative days from Day 0 to Day 12. (Ai) Percentage body weight change (ii) Disease activity index (iii) Occult blood (iv) Feces score. (Bi-ii) Colon length (iii) Colon weight/length ratio. (C) Representative microscopic image of H&E-stained colon tissues of the experimental groups (4× magnification). All data are expressed as means±SEM. *p<0.05, **p<0.01 ***p<0.001 and ****p<0.0001.

FIGS. 17A-C Effects of Mrgprx2 ligands on the clinical parameters of DSS-induced colitis in Mrgprb2 KO mice. Mrgprb2^{-/-} mice were subjected to 2% DSS (mixed in drinking water) and water (without DSS) for Day 0 to Day 5 and DSS was replaced with normal water cycle (ad libitum) from Day 5 to Day 12. P17, P17 analogues, and GE1111 were i.p. injected every alternative days from Day 0 to Day 12. (Ai) Percentage body weight change (ii) Disease activity index (iii) Occult blood (iv) Feces score. (Bi-ii) Colon length (iii) Colon weight/length ratio. (C) Representative microscopic image of H&E-stained colon tissues of the experimental groups (4× magnification). All data are expressed as means±SEM. *p<0.05, **p<0.01 ***p<0.001 and ****p<0.0001.

FIG. 18 Mrgprb2 and its ligands on DSS-induced colitis severity

FIG. 19 Schematic diagram of the effects of mrgprb2 and its ligands on DSS-induced colitis severity.

FIG. 20 Western blot analysis of LAD2 cells treated with Mrgprx2 ligands. (A) Blot images showing pERK1/2, ERK1/2, GAPDH and STIM1 protein expression.

FIG. 21 Structure of GE1111.

### 4.1 DEFINITION

The following definitions are more general terms used throughout the present application: “Modulating” MRGPRX2 means that the compound interacts with MRGPRX2 or MRGPRX2 orthologs in a manner such that it functions as an inverse agonist to the receptor, and/or as a competitive antagonist to the receptor. In one embodiment, such modulation is partially or fully selective against other MRGPRs, such as MRGPR X1, X3 and/or X4.

“MRGPR” refers to one or more of the Mas-related G protein coupled receptors, which are a group of orphan receptors with limited expression in very specialized tissues (e.g., in mast cells and dorsal root ganglia) and barrier tissues. There are eight related receptors in this class expressed in humans, only 4 of which have readily identifiable orthologs in other species (i.e., MRGPR D, E, F and G). The other four receptors (MRGPR X1, X2, X3 and X4) have no single ortholog, based on homology, in non-human species. Among rodent receptors, mouse mrgprb2 and rat mrgprb3 correspond functionally to human MRGPRX2 on mast cells.

“MRGPRX2,” also referred to as “MRGX2,” or “MGRG3,” refers to a member of the MRGPR family that is expressed on mast cells and capable of mediating IgE independent activation (e.g., mast cell degranulation) in response to ligand binding.

“Isomer” is used herein to encompass all chiral, diastereomeric or racemic forms of a structure (also referred to as a stereoisomer, as opposed to a structural or positional isomer), unless a particular stereochemistry or isomeric form is specifically indicated. Such compounds can be enriched or resolved optical isomers at any or all asymmetric atoms as are apparent from the depictions, at any degree of enrichment. Both racemic and diastereomeric mixtures, as well as the individual optical isomers can be synthesized so as to be substantially free of their enantiomeric or diastereomeric partners, and these are all within the scope of certain embodiments of the invention. The isomers resulting from the presence of a chiral center comprise a pair of nonsuperimposable-isomers that are called “enantiomers.” Single enantiomers of a pure compound are optically active (i.e., they are capable of rotating the plane of plane polarized light and designated R or S).

“Isolated optical isomer” means a compound which has been substantially purified from the corresponding optical isomer(s) of the same formula. For example, the isolated isomer may be at least about 80%, at least 80% or at least 85% pure by weight. In other embodiments, the isolated isomer is at least 90% pure or at least 98% pure, or at least 99% pure by weight.

“Substantially enantiomerically or diastereomerically” pure means a level of enantiomeric or diastereomeric enrichment of one enantiomer with respect to the other enantiomer or diastereomer of at least about 80%, and more specifically in excess of 80%, 85%, 90%, 95%, 98%, 99%, 99.5% or 99.9%.

The terms “racemate” and “racemic mixture” refer to an equal mixture of two enantiomers. All compounds with an asterisk (*) adjacent to a tertiary or quaternary carbon are optically active isomers, which may be purified from the respective racemate and/or synthesized by appropriate chiral synthesis.

A “hydrate” is a compound that exists in combination with water molecules. The combination can include water in stoichiometric quantities, such as a monohydrate or a dihydrate, or can include water in random amounts. As the term is used herein a “hydrate” refers to a solid form; that is, a compound in a water solution, while it may be hydrated, is not a hydrate as the term is used herein.

A “solvate” is similar to a hydrate except that a solvent other than water is present. For example, methanol or ethanol can form an “alcoholate”, which can again be stoichiometric or non-stoichiometric. As the term is used herein a “solvate” refers to a solid form; that is, a compound in a solvent solution, while it may be solvated, is not a solvate as the term is used herein.

“Isotope” refers to atoms with the same number of protons but a different number of neutrons, and an isotope of a compound of structure (I) includes any such compound wherein one or more atoms are replaced by an isotope of that atom. For example, carbon 12, the most common form of carbon, has six protons and six neutrons, whereas carbon 13 has six protons and seven neutrons, and carbon 14 has six protons and eight neutrons. Hydrogen has two stable isotopes, deuterium (one proton and one neutron) and tritium (one proton and two neutrons). While fluorine has a number of isotopes, fluorine-19 is longest-lived. Thus, an isotope of a compound having the structure of structure (I) includes, but not limited to, compounds of structure (I) wherein one or more carbon 12 atoms are replaced by carbon-13 and/or carbon-14 atoms, wherein one or more hydrogen atoms are replaced with deuterium and/or tritium, and/or wherein one or more fluorine atoms are replaced by fluorine-19.

“Salt” generally refers to an organic compound, such as a carboxylic acid or an amine, in ionic form, in combination with a counter ion. For example, salts formed between acids in their anionic form and cations are referred to as “acid addition salts”. Conversely, salts formed between bases in the cationic form and anions are referred to as “base addition salts.”

The term “subject,” as used herein, refers to any animal. In certain embodiments, the subject is a mammal. In certain embodiments, the term “subject”, as used herein, refers to a human (e.g., a man, a woman, or a child).

The terms “administer,” “administering,” or “administration,” as used herein refers to implanting, absorbing, ingesting, injecting, or inhaling, the disclosed polymer or compound.

The terms “treat” or “treating,” as used herein, refers to partially or completely alleviating, inhibiting, ameliorating, and/or relieving the disease or condition from which the subject is suffering.

The terms “effective amount” and “therapeutically effective amount,” as used herein, refer to the amount or concentration of a biologically active agent conjugated to the disclosed polymer, or amount or concentration of the polymer, that, when administered to a subject, is effective to at least partially treat a condition from which the subject is suffering.

The term “pharmaceutically acceptable” refers to an agent that has been approved for human consumption and is generally non-toxic. For example, the term “pharmaceutically acceptable salt” refers to nontoxic inorganic or organic acid and/or base addition salts (see, e.g., Lit et al., Salt Selection for Basic Drugs, Int. J. Pharm., 33, 201-217, 1986) (incorporated by reference herein).

As used herein, the term “autoimmune disorder”, or “inflammatory disorder” means a disease or disorder arising from and/or directed against an individual's own tissues or organs, or a co-segregate or manifestation thereof, or resulting condition therefrom. Typically, various clinical and laboratory markers of autoimmune diseases may exist including, but not limited to, hypergammaglobulinemia, high levels of autoantibodies, antigen-antibody complex deposits in tissues, clinical benefit from corticosteroid or immunosuppressive treatments, and lymphoid cell aggregates in affected tissues.

### 5. DETAILED DESCRIPTION

The mast cell receptor Mrgprb2, a mouse orthologue of human Mrgprx2, is known as an inflammatory receptor and its elevated expression is associated with various diseases such as ulcerative colitis. We aimed to elucidate the role of Mrgprb2/x2 and the effect of its ligands on a chemically induced murine colitis model. We showed that in Mrgprb2−/− mice, there is a differential regulation of cytokine releases in the blood plasma and severe colonic damages after DSS treatment. Unexpectedly, we demonstrated that known Mrgprb2/x2 agonists (peptide P17, P17 analogues and CST-14) and antagonist (GE1111) similarly increased the survival rate of WT mice subjected to 4% DSS-induced colitis, ameliorated the colonic damages of 2.5% DSS-induced colitis, restored major protein mRNA expression involved in colon integrity, reduced CD68+ and F4/80+immune cell infiltration and restored cytokine levels. Collectively, our findings highlight the eminent role of Mrpgrb2/x2 in conferring a beneficial effect in the colitis model, and this significance is demonstrated by the heightened severity of colitis with altered cytokine releases and inflammatory immune cell infiltration observed in the Mrgprb2 knockout mice. Elevated expression of Mrgprb2 in WT colitis murine models may represent the organism's adaptive protective mechanism since Mrgprb2 knockout results in severe colitis. On the other hand, both agonist and antagonist of Mrgprb2 analogously mitigated the severity of colitis in DSS-induced colitis model by altering Mrgprb2 expression, immune cell infiltration and inflammatory cytokine releases.

In certain embodiments, the disclosure provides a pharmaceutical composition comprising a compound of structure (I), or a pharmaceutically acceptable salt, isomer, hydrate, solvate or isotope thereof, together with at least one pharmaceutically acceptable carrier, diluent, or excipient. For example, the active compound will usually be mixed with a carrier, or diluted by a carrier, or enclosed within a carrier which can be in the form of an ampoule, capsule, sachet, paper, or other container. When the active compound is mixed with a carrier, or when the carrier serves as a diluent, it can be solid, semi-solid, or liquid material that acts as a vehicle, excipient, or medium for the active compound. The active compound can be adsorbed on a granular solid carrier, for example contained in a sachet. Some examples of suitable carriers are water, salt solutions, alcohols, polyethylene glycols, polyhydroxyethoxylated castor oil, peanut oil, olive oil, gelatin, lactose, terra alba, sucrose, dextrin, magnesium carbonate, sugar, cyclodextrin, amylose, magnesium stearate, talc, gelatin, agar, pectin, acacia, stearic acid, or lower alkyl ethers of cellulose, silicic acid, fatty acids, fatty acid amines, fatty acid monoglycerides and diglycerides, pentaerythritol fatty acid esters, polyoxyethylene, hydroxymethylcellulose, and polyvinylpyrrolidone. Similarly, the carrier or diluent can include any sustained release material known in the art, such as glyceryl monostearate or glyceryl distearate, alone or mixed with a wax.

As used herein, the term “pharmaceutical composition” refers to a composition containing one or more of the compounds described herein, or a pharmaceutically acceptable isomer, racemate, hydrate, solvate, isotope or salt thereof, formulated with a pharmaceutically acceptable carrier, which can also include other additives, and manufactured or sold with the approval of a governmental regulatory agency as part of a therapeutic regimen for the treatment of disease in a mammal. Pharmaceutical compositions can be formulated, for example, for oral administration in unit dosage form (e.g., a tablet, capsule, caplet, gelcap, or syrup); for topical administration (e.g., as a cream, gel, lotion, or ointment); for intravenous administration (e.g., as a sterile solution free of particulate emboli and in a solvent system suitable for intravenous use); for administration to a pediatric subject (e.g., solution, syrup, suspension, elixir, powder for reconstitution as suspension or solution, dispersible/effervescent tablet, chewable tablet, lollipop, freezer pops, troches, oral thin strips, orally disintegrating tablet, orally disintegrating strip, and sprinkle oral powder or granules); or in any other formulation described herein, including spray dry dispersions (e.g., a single-phase, amorphous molecular dispersion of a compound as described herein in a polymer matrix). Conventional procedures and ingredients for the selection and preparation of suitable formulations are described, for example, in Remington: The Science and Practice of Pharmacy, 21st Ed., Gennaro, Ed., Lippencott Williams & Wilkins (2005) and in The United States Pharmacopeia: The National Formulary (USP 36 NF31), published in 2013.

In certain embodiments, the disclosure provides a pharmaceutical composition comprising a compound of structure (I), or a pharmaceutically acceptable salt, isomer, hydrate, solvate or isotope thereof, wherein the compound, or a pharmaceutically acceptable salt, isomer, hydrate, solvate or isotope thereof, is in an amorphous form. In other embodiments the compound, or a pharmaceutically acceptable salt, isomer, hydrate, solvate or isotope thereof, is micronized. In still other embodiments the compound, or a pharmaceutically acceptable salt, isomer, hydrate, solvate or isotope thereof, is crystalline.

In certain embodiments, the disclosure provides a pharmaceutical composition comprising a compound of structure (I), or a pharmaceutically acceptable salt, isomer, hydrate, solvate or isotope thereof, wherein the compound, or the pharmaceutically acceptable salt, isomer, hydrate, solvate or isotope thereof, has a distribution of particle sizes. For example, in some embodiments a D₅₀ particle size distribution of the compound of structure (I), or a pharmaceutically acceptable salt, isomer, hydrate, solvate or isotope thereof, ranges from about 500 nm to about 500 μm. In other embodiments, the D₅₀ particle size distribution of the compound of structure (I), or a pharmaceutically acceptable salt, isomer, hydrate, solvate or isotope thereof, ranges from about 500 nm to about 1 μm, or from about 1 am to about 50 μm, or from about 50 nm to about 100 μm, or from about 100 nm to about 150 μm. In still other embodiments, the D₅₀ particle size distribution of the compound of structure (I), or a pharmaceutically acceptable salt, isomer, hydrate, solvate or isotope thereof, ranges from about 1 μm to about 10 μm, or from about 2 nm to about 8 μm, or from about 3 nm to about 7 μm.

In certain embodiments, a D₉₀ particle size distribution of the compound of structure (I), or a pharmaceutically acceptable salt, isomer, hydrate, solvate or isotope thereof, ranges from about 1 μm to about 1000 μm. In other embodiments, the D₉₀ particle size distribution of the compound of structure (I), or a pharmaceutically acceptable salt, isomer, hydrate, solvate or isotope thereof, ranges from about 2 μm to about 100 μm, or from about 100 nm to about 200 μm, or from about 200 nm to about 300 μm, or from about 300 nm to about 400 μm, or from about 400 nm to about 500 μm, or from about 500 μm to about 600 μm, or from about 600 μm to about 700 μm, or from about 700 μm to about 800 μm, or from about 800 μm to about 900 μm, or from about 900 μm to about 1000 μm. In still other embodiments, the D₉₀ particle size distribution of the compound of structure (I), or a pharmaceutically acceptable salt, isomer, hydrate, solvate or isotope thereof, ranges from about 5 μm to about 25 μm, or from about 7 nm to about 23 μm, or from about 9 nm to about 21 μm, or from about 11 nm to about 19 μm.

In some embodiments, the pharmaceutical composition comprising a compound of structure (I), or a pharmaceutically acceptable salt, isomer, hydrate, solvate or isotope thereof, with at least one pharmaceutically acceptable carrier, diluent, or excipient further comprises a second therapeutic agent.

As used herein, the term “pharmaceutically acceptable carrier” refers to any ingredient other than the disclosed compounds, or a pharmaceutically acceptable isomer, racemate, hydrate, solvate, isotope or salt thereof (e.g., a carrier capable of suspending or dissolving the active compound) and having the properties of being nontoxic and non-inflammatory in a patient. Excipients may include, for example: antiadherents, antioxidants, binders, coatings, compression aids, disintegrants, dyes (colors), emollients, emulsifiers, fillers (diluents), film formers or coatings, flavors, fragrances, glidants (flow enhancers), lubricants, preservatives, printing inks, sorbents, suspending or dispersing agents, sweeteners, or waters of hydration. Exemplary excipients include, but are not limited to: butylated hydroxytoluene (BHT), calcium carbonate, calcium phosphate (dibasic), calcium stearate, croscarmellose, crosslinked polyvinyl pyrrolidone, citric acid, crospovidone, cysteine, ethylcellulose, gelatin, hydroxypropyl cellulose, hydroxypropyl methylcellulose, lactose, magnesium stearate, maltitol, mannitol, methionine, methylcellulose, methyl paraben, microcrystalline cellulose, polyethylene glycol, polyvinyl pyrrolidone, povidone, pregelatinized starch, propyl paraben, retinyl palmitate, shellac, silicon dioxide, sodium carboxymethyl cellulose, sodium citrate, sodium starch glycolate, sorbitol, starch (corn), stearic acid, stearic acid, sucrose, talc, titanium dioxide, vitamin A, vitamin E, vitamin C, and xylitol.

In certain embodiments, the disclosure provides a pharmaceutical composition comprising a compound of structure (I), or a pharmaceutically acceptable salt, isomer, hydrate, solvate or isotope thereof, and at least one polymeric carrier. In some embodiments, the pharmaceutical composition is in the form of an amorphous solid dispersion including at least one polymeric carrier.

In some embodiments, the polymeric carrier comprises at least one selected from the group consisting of an enteric polymer, a hydrophilic polymer, a surfactant, an amphiphilic polymer, and combinations thereof.

In some embodiments, the pharmaceutical composition contains a compound of structure (I), or a pharmaceutically acceptable salt, isomer, hydrate, solvate or isotope thereof, and a polymeric carrier comprising at least one selected from the group consisting of a methacrylate polymer, a hydroxylpropyl methyl cellulose phthalate (HPMCP) polymer, a hydroxypropyl methyl cellulose acetate-succinate (HPMCAS) polymer, a cellulose acetylate phthalate (CAP) polymer, a starch, a sodium carboxymethyl cellulose polymer, a sodium alginate polymer, a polyethylene glycol (PEG), a polyvinyl pyrollidone (PVP), a hydroxy propyl methyl cellulose (HPMC) polymer, a polyvinvyl alcohol (PVA), a beta-cyclodextrin polymer, a mannitol polymer, a chitosan polymer, a carrageenan polymer, a hydroxypropylcellulose (HPC) polymer, a polyethylene-polypropylene glycol, a lecithin, a bile salt, a lauroyl polyoxyl-32 μglyceride, a polyethylene oxide (PEO)/polypropylene glycol (PPG) copolymer, a PEG-modified starch, a vinyl acetate/vinylpyrrolidone random copolymer, a polyacrylic acid, a polyacrylate, and a polyvinyl caprolactam-polyvinyl acetate-polyethylene glycol (PCL-PVAc-PEG) graft copolymer.

In some embodiments, the pharmaceutical composition contains a compound of structure (I), or a pharmaceutically acceptable salt, isomer, hydrate, solvate or isotope thereof, and a polymeric carrier comprising at least one selected from the group consisting of a hydroxy propyl methyl cellulose (HPMC) polymer, a hydroxypropyl methyl cellulose acetate-succinate (HPMCAS) polymer, a polyvinyl pyrollidone (PVP) and a hydroxypropylcellulose (HPC) polymer. In more specific embodiments, the polymeric carrier comprises a hydroxypropyl methyl cellulose acetate-succinate (HPMCAS) polymer. For example, in some embodiments the polymeric carrier comprises HPMCAS-MG.

In some embodiments, the pharmaceutical composition comprises a compound of structure (I), or a pharmaceutically acceptable salt, isomer, hydrate, solvate or isotope thereof, and at least one polymeric carrier, such that a mass ratio of the compound to the polymeric carrier that ranges from about 1:100 to about 50:1. For example, in some embodiments the mass ratio of the compound to the polymeric carrier ranges from about 1:100 to about 1:50; or from about 1:50 to about 1:10; or from about 1:10 to about 1:1, or from about 1:1 to about 10:1, or from about 10:1 to about 20:1, or from about 20:1 to about 50:1. In other embodiments, the mass ratio of the compound to the polymer carrier ranges from about 1:1 to about 1:5.

In some embodiments, the pharmaceutical composition comprises a compound of structure (I), or a pharmaceutically acceptable salt, isomer, hydrate, solvate or isotope thereof, and at least one polymeric carrier, such that a weight percent of the compound ranges from about 2% to about 70% relative to a combined mass of the compound and the polymeric carrier. In other embodiments, the weight percent of the compound ranges from about 2% to about 10%, or from about 10% to about 20%, or from about 15% to about 25%, or from about 20% to about 30%, or from about 30% to about 40%, or from about 40% to about 50%, or from about 50% to about 60%, or from about 60% to about 70%, relative to a combined mass of the compound and the polymeric carrier.

In certain embodiments, the disclosure provides a pharmaceutical composition further comprising at least one pharmaceutically acceptable excipient. For example, in some embodiments the pharmaceutical composition comprises a compound of structure (I), or a pharmaceutically acceptable salt, isomer, hydrate, solvate or isotope thereof, at least one polymeric carrier as described above, and at least one pharmaceutically acceptable excipient. Pharmaceutically acceptable excipients may include, for example, fillers, binders, disintegrants, surfactants, lubricants, glidants, capsule shells, and mixtures thereof. Fillers may include, for example, mannitol, a microcrystalline cellulose (MCC), a silicified microcrystalline cellulose (SMCC) and mixtures thereof. Binders may include, for example, a hydroxypropylmethylcellulose (HPMC), a povidone, and mixtures thereof. Disintegrants may include, for example, a sodium starch glycolate (SSG), a croscarmellose sodium (CCS), and mixtures thereof. Surfactants may include, for example, sodium lauryl sulfate (SLS), a polaxamer, and mixtures thereof. Lubricants may include, for example, magnesium stearant (MS), sodium stearyl fumarate (SSF), and mixtures thereof. Glidants may include, for example, silicon dioxide, talc, and mixtures thereof. Capsule shells may include, for example, an HPMC capsule or a gelatin capsule. Surfactants may include, for example, sodium lauryl sulfate (SLS).

In some embodiments, the pharmaceutical composition comprises at least one solvent. Solvents may include, for example, water and organic solvents (such as dimethylsulfoxide and the like).

Pharmaceutically acceptable base addition salts of compounds of the disclosure include, for example, metallic salts including alkali metal, alkaline earth metal, and transition metal salts such as, for example, calcium, magnesium, potassium, sodium, and zinc salts. Pharmaceutically acceptable base addition salts also include organic salts made from basic amines such as, for example, N,N′dibenzylethylenediamine, chloroprocaine, choline, diethanolamine, ethylenediamine, meglumine (N-methylglucamine), and procaine.

Pharmaceutically acceptable acid addition salts may be prepared from an inorganic acid or from an organic acid. Examples of inorganic acids include hydrochloric, hydrobromic, hydriodic, nitric, carbonic, sulfuric, and phosphoric acids. Appropriate organic acids may be selected from aliphatic, cycloaliphatic, aromatic, aromatic aliphatic, heterocyclic, carboxylic, and sulfonic classes of organic acids, examples of which include formic, acetic, propionic, succinic, glycolic, gluconic, lactic, malic, tartaric, citric, ascorbic, glucuronic, maleic, fumaric, pyruvic, aspartic, glutamic, benzoic, anthranilic, 4-hydroxybenzoic, phenylacetic, mandelic, hippuric, malonic, oxalic, embonic (pamoic), methanesulfonic, ethanesulfonic, benzenesulfonic, panthothenic, trifluoromethanesulfonic, 2-hydroxyethanesulfonic, p-toluenesulfonic, sulfanilic, cyclohexylaminosulfonic, stearic, alginic, α-hydroxybutyric, salicylic, galactaric, and galacturonic acid.

The compounds of the disclosure (i.e., compounds of structure (I) and embodiments thereof), or their pharmaceutically acceptable salts may contain one or more centers of geometric asymmetry and may thus give rise to enantiomers, diastereomers, and other stereoisomeric forms that are defined, in terms of absolute stereochemistry, as (R)- or (S)- or, as (D)- or (L)- for amino acids. Embodiments thus include all such possible isomers, as well as their racemic and optically pure forms.

Optically active (+) and (−), (R)- and (S)-, or (D)- and (L)-isomers may be prepared using chiral synthons or chiral reagents, or resolved using conventional techniques, for example, chromatography and fractional crystallization. Conventional techniques for the preparation/isolation of individual enantiomers include chiral synthesis from a suitable optically pure precursor or resolution of the racemate (or the racemate of a salt or derivative) using, for example, chiral high pressure liquid chromatography (HPLC). When the compounds described herein contain olefinic double bonds or other centers of geometric asymmetry, and unless specified otherwise, it is intended that the compounds include both E and Z geometric isomers. Likewise, all tautomeric forms are also included.

Although pharmaceutically unacceptable salts are not generally useful as medicaments, such salts may be useful, for example as intermediates in the synthesis of compounds having the structure (1), for example in their purification by recrystallization.

The formulations can be mixed with auxiliary agents which do not deleteriously react with the active compounds. Such additives can include wetting agents, emulsifying and suspending agents, salt for influencing osmotic pressure, buffers and/or coloring substances, preserving agents, sweetening agents, or flavoring agents. The compositions can also be sterilized if desired.

In another embodiment, the method of treating a subject having a MRGPRX2 dependent condition (e.g., an inflammatory or autoimmune disorder) described herein further comprises administering to the subject a pharmaceutically effective amount of a second therapeutic agent.

In certain embodiments, the second therapeutic agent is a MRGPRX2 binding molecule such as a agonist. In certain embodiments, the second therapeutic agent is peptide P17, P17 analogues or CST-14.

The second therapeutic agent may be administered simultaneously, separately, or sequentially with the compounds of the present disclosure. If administered simultaneously, the second therapeutic agent and compound of the present disclosure may be administered in separate dosage forms or in the same dosage form.

As used herein, the phrase “MRGPRX2 dependent condition” means a condition where the activation, over sensitization, or desensitization of MRGPRX2 or its orthologs by a natural or synthetic ligand initiates, mediates, sustains, or augments a pathological condition. For example, it is known that some cationic peptidergic drugs cause pseudo-allergic reactions in patients where MRGPRX2 is sensitive to (or activated by) secretagogues, cationic peptidergic drugs, including icatibant, leuprolide, or ganirelix, neutral and anionic peptidergic drugs (e.g., exenatide, glucagon, liraglutide, enfuviritide, colistimethate), non-steroidal agonist (atracurium mivacurium), non-steroidal antagonist drugs, neuropeptides, and antimicrobial peptides. Moreover, overexpression of MRGPRX2 and/or overactivity MRGPRX2 may also render mast cells more susceptible to activation by endogenous and/or exogenous ligands. Without limited by theory, it is to be understood that by modulating MRGPRX2, pseudo-allergic reactions, itch, pain, inflammatory or autoimmune disorders can be eased.

In some embodiments, the MRGPRX2 dependent condition is a condition that is caused by IgE independent activation of MRGPRX2 or its orthologs. IgE independent activation of MRGPRX2 is capable of inducing mast cell degranulation. For example, IgE independent mast cell activation is associated with some cases of chronic urticaria and other mast cell mediated conditions, which are not responsive to current anti-IgE or antihistamine therapies. Thus, the compounds of the present disclosure may be used for treating an MRGPRX2 dependent condition caused by IgE independent activation of MRGPRX2 and that would benefit from modulating MRGPRX2.

In some embodiments, the MRGPRX2 dependent condition is an itch associated condition, a pain associated condition, a cancer associated condition, a pseudo-allergic reaction, or an autoimmune or inflammatory disorder in humans or other mammals.

In one embodiment, the method of present disclosure is provided to treat an autoimmune disorder, autoinflammatory disease, celiac disease, chronic prostatitis, diverticulitis, glomerulonephritis, hidradenitis suppurativa, hypersensitivities, intestinal disorder, epithelial intestinal disorder, inflammatory bowel disease, irritable bowel syndrome, Crohn's Disease, ulcerative colitis, lupus erythematous, interstitial cystitis, otitis, pelvic inflammatory disease, endometrial pain, reperfusion injury, rheumatic fever, rheumatoid arthritis, sarcoidosis, transplant rejection, psoriasis, lung inflammation, chronic obstructive pulmonary disease.

In certain embodiments, the composition is used for the treatment of inflammatory disease is (e.g., Crohn's disease, inflammatory bowel disease, ulcerative colitis, pancreatitis, hepatitis, appendicitis, gastritis, diverticulitis, celiac disease, food intolerance, enteritis, ulcer, gastroesophageal reflux disease (GERD), psoriatic arthritis, psoriasis, and rheumatoid arthritis) in a subject in need thereof.

The route of administration can be any route which effectively transports the active compound of the disclosure to the appropriate or desired site of action, such as oral, nasal, pulmonary, buccal, subdermal, intradermal, transdermal, or parenteral, including intravenous, subcutaneous and/or intramuscular. In one embodiment, the route of administration is oral. In another embodiment, the route of administration is topical.

Dosage forms can be administered once a day, or more than once a day, such as twice or thrice daily. Alternatively, dosage forms can be administered less frequently than daily, such as every other day, or weekly, if found to be advisable by a prescribing physician or drug's prescribing information. Dosing regimens include, for example, dose titration to the extent necessary or useful for the indication to be treated, thus allowing the patient's body to adapt to the treatment, to minimize or avoid unwanted side effects associated with the treatment, and/or to maximize the therapeutic effect of the present compounds. Other dosage forms include delayed or controlled-release forms. Suitable dosage regimens and/or forms include those set out, for example, in the latest edition of the Physicians' Desk Reference, incorporated herein by reference.

Proper dosages for pediatric patients can be determined using known methods, including weight, age, body surface area, and models such as Simcyp Pediatric Simulation modeling (CERTARA, Princeton, N.J.) which can be used to establish a pharmacokinetic approach for dosing that takes into account patient age, ontogeny of the clearance pathways to eliminate a compound. In one embodiment, the dosage form is formulated to provide a pediatric dose from about 30% to about 100% of an adult dose, or about 35%, 40%, 45%, 50%, 55%, 60%, 65%, 70%, 75%, 80%, 85%, 90%, 95%, or 100% of an adult dose.

In one embodiment, the disclosure provides an oral pharmaceutical composition comprising a compound of structure (I), or a pharmaceutically acceptable salt, isomer, hydrate, solvate or isotope thereof, together with at least one pharmaceutically acceptable oral carrier, diluent, or excipient. For example, the oral pharmaceutical composition is provided to treat UC, wherein the dosage regimen is, for example, once a day.

In another embodiment, there are provided methods of making a composition of a compound described herein including formulating a compound of the disclosure with a pharmaceutically acceptable carrier or diluent. In some embodiments, the pharmaceutically acceptable carrier or diluent is suitable for oral administration. In some such embodiments, the methods can further include the step of formulating the composition into a tablet or capsule. In other embodiments, the pharmaceutically acceptable carrier or diluent is suitable for parenteral administration. In some such embodiments, the methods further include the step of lyophilizing the composition to form a lyophilized preparation. In some embodiments, the composition is formulated into a pediatric dosage form suitable for treating a pediatric subject.

In certain embodiments, the disclosure provides a compound having structure (I), or a pharmaceutically acceptable salt, isomer, hydrate, solvate or isotope thereof. Such compounds can be synthesized using standard synthetic techniques known to those skilled in the art. For example, compounds of the present disclosure can be synthesized using appropriately modified synthetic procedures set forth in the following Examples and Reaction Schemes.

To this end, the reactions, processes, and synthetic methods described herein are not limited to the specific conditions described in the following experimental section, but rather are intended as a guide to one with suitable skill in this field. For example, reactions may be carried out in any suitable solvent, or other reagents to perform the transformation[s] necessary. Generally, suitable solvents are protic or aprotic solvents which are substantially non-reactive with the reactants, the intermediates or products at the temperatures at which the reactions are carried out (i.e., temperatures which may range from the freezing to boiling temperatures). A given reaction may be carried out in one solvent or a mixture of more than one solvent. Depending on the particular reaction, suitable solvents for a particular work-up following the reaction may be employed.

All reagents, for which the synthesis is not described in the experimental part, are either commercially available, or are known compounds or may be formed from known compounds by known methods by a person skilled in the art. The compounds and intermediates produced according to the methods of the disclosure may require purification. Purification of organic compounds is well known to a person skilled in the art and there may be several ways of purifying the same compound. In some cases, no purification may be necessary. In some cases, the compounds may be purified by crystallization. In some cases, impurities may be stirred out using a suitable solvent. In some cases, the compounds may be purified by chromatography, particularly flash column chromatography, using purpose-made or prepacked silica gel cartridges and eluents such as gradients of solvents such as heptane, ether, ethyl acetate, acetonitrile, ethanol and the like. In some cases, the compounds may be purified by preparative HPLC using methods as described.

Purification methods as described herein may provide compounds of the present disclosure which possess a sufficiently basic or acidic functionality in the form of a salt, such as, in the case of a compound of the present disclosure which is sufficiently basic, a trifluoroacetate or formate salt, or, in the case of a compound of the present disclosure which is sufficiently acidic, an ammonium salt. A salt of this type can either be transformed into its free base or free acid form, respectively, by various methods known to a person skilled in the art, or be used as salts in subsequent biological assays.

It is to be understood that the specific form of a compound of the present disclosure as isolated and as described herein is not necessarily the only form in which said compound can be applied to a biological assay in order to quantify the specific biological activity.

Chemical names were generated using the ChemDraw naming software (Version 17.0.0.206) by PerkinElmer Informatics, Inc. In some cases, generally accepted names of commercially available reagents were used in place of names generated by the naming software.

### 6. EXAMPLES

### 6.1 Materials and Methods

### 6.1.1 Animals

C57BL/6N and Mrgprb2−/− adult male mice, about 6 to 8 weeks old, were obtained from The Centre for Comparative Medicine Research (CCMR) of the University of Hong Kong (AAALAC International accredited). This study was conducted in strict accordance with the recommendations stated in the Guide for the Care and Use of Laboratory Animals of the National Institutes of Health. Protocols of the study were approved by the Committee on the Use of Live Animals in Teaching and Research (CULATR 5417-20) of the University of Hong Kong. All animal procedures were performed under ketamine/xylazine anesthesia.

### 6.1.2 Induction of Colitis in Mice by DSS

C57BL/6N and Mrgprb2−/− adult mice (6-8 weeks old) were obtained from CMMR and housed in cages for acclimatation for a week. C57BL/6N and Mrgprb2−/− mice were divided into four groups (N=5-6) (2% DSS in drinking water and water control). Controls were fed with regular water and the treatment groups were exposed to 2% DSS for 5 days (Day 0 to Day 5) and regular water till Day 12. DSS-Colitis Grade (36,000-50,000 MW) (MP Biomedicals, Santa Ana, CA) was used in this study. During experiments, these mice were given anaesthesia to harvest blood and organs for other biochemical and histology analysis. For the survival study with Mrgprb2/x2 agonists, C57BL/6N mice were grouped into 3 μgroups (N=10) and fed with 4% DSS for 5 days (Day 0 to 5) and replaced with regular drinking water for the rest of the experiment. Meanwhile, Mrgprb2/x2 ligands were given to the animals every alternative day via i.p. injection 100 μL with 10 μg/kg mice. For the treatment study with Mrgprb2/x2 ligands, C57BL/6N mice were divided into 7 groups (N=8 per group). Six groups were receiving 2.5% DSS dissolved in drinking water for 5 days (Day 0 to Day 5) and regular water till Day 12 and the control group was receiving water from Day 0 to Day 12. These 6 groups exposed to 2.5% DSS were treated with Mrgprb2/x2 ligands or saline for every alternative day till Day 12. The treatment groups are saline control (Saline+DSS), agonists: P17 (150 μg/kg P17+DSS), P17*A4 (150 μg/kg P17*A4+DSS), P17*A7 (150 μg/kg P17*A7+DSS), CST-14 (15 μg/kg CST-14+DSS), and the antagonist GE1111 (300 μg/kg GE1111+DSS). Hemoccult Sensa Single Slides Rapid Diagnostic Test Kit (Beckman Coulter) were used to detect the blood in stool. Treatment groups were anaesthetized to harvest blood and scarified to harvest organs for various biochemical and histology analysis.

6.1.3 FITC dextran permeability assay

C57BL/6N mice were divided into 7 μgroups (N=4-8 per group). Six groups were receiving 2.5% DSS dissolved in drinking water for 5 days (Day 0 to Day 5) and regular water till Day 12 and the control group was receiving water from Day 0 to Day 12. These 6 μgroups exposed to 2.5% DSS were treated with Mrgprb2/x2 ligands or saline for every alternative day till Day 12. The treatment groups are saline control (Saline+DSS), agonists: P17 (P17+DSS), P17*A4 (P17*A4+DSS), P17*A7 (P17*A7+DSS), CST-14 (CST-14+DSS), and the antagonist GE1111 (GE1111+DSS). Before the start of the FITC dextran assay, food and water were withdrawn for 4 hr, and mice were subsequently gavage fed with 4 kDa FITC-Dextran (Merck) solution of 60 mg/100 μg body weight. Blood plasma was collected 4 hr post gavage feeding of FITC-dextran. Fluorescent intensity was measured with excitation 485 nm and emission 530 nm using PerkinElmer Victor X4. Serial dilutions of FITC-dextran in PBS were used to calculate standard curve.

6.1.4 Real-time quantitative RT-PCR

To assess the gene expression profile in colon, distal colons were used to isolate total RNA using Trizol. Total RNA was precipitated for 30 min in 20° C. with isopropanol and glycogen. The precipitated RNA was then washed with 75% ethanol and resuspended in RNase-free water for first strand cDNA synthesis following the protocol from HiScript® II Q RT SuperMix (Vazyme biotech co., ltd, China). cDNA harvested were quantified by quantitative PCR (7300 Real-Time PCR System, Applied Biosystems, Forster City, CA). The expression of genes was examined using ChamQ™ SYBR Color qPCR Master Mix as specified by the manufacturer protocol. All primer sequences are shown in Supplementary Table S1. ₂-AAc^{t} method [34] was employed for data analysis with the internal control, glyceraldehyde-3-phosphate dehydrogenase (gapdh).

### 6.1.5 Immunohistochemistry

Distal colons were isolated from mice from all the groups (WT+Water, WT+2% DSS, Mrgprb2−/−+Water, Mrgprb2−/−+2% DSS, Water+Saline, 2.5% DSS+Saline, 2.5% DSS+P17, 2.5% DSS+P17*A4, 2.5% DSS+P17*A7, 2.5% DSS+CST-14 and 2.5% DSS+GE1111). These swiss-rolled colons were fixed in 10% formaldehyde, dehydrated with graded concentration of ethanol, embedded in paraffin, and sectioned (5 □m). Immunostaining was performed with a Leica Bond-Max automatic immunostainer (Leica Bannockburn, IL) according to the recommended procedure using rabbit anti-claudin 1 antibody (1:200 dilution; ab15098, Abcam), anti-CD68 (1:200 dilution; abl25212, Abcam), and anti-F4/80 (1:200 dilution; ab100790, Abcam) and anti-COX2 (1:200 dilution; ab15191, Abcam). Images were captured with Nikon Eclipse Ni-U upright microscope (Nikon H550L) [35]. Statistical comparisons were made between the number of positive cells or optical intensity in these tissues between control and treatments.

6.1.6 Hematoxylin and eosin (H&E) staining

H&E staining was performed using Leica ST5020 Multistainer according to recommended procedures. Paraffin sections (5 μm) were dewaxed and rehydrated in degressive concentrations of ethanol. After washing with distilled water (diH₂O), the sections were stained with hematoxylin for 10 min, followed by rinsing with tap water for 1 min and staining with eosin for another 1 min. H&E-stained sections were washed by diH₂O and allowed to air dry before mounted with Histomount (Invitrogen). Images of colon tissues were captured using Nikon Eclipse Ni-U upright microscope (Nikon H550L) [35].

### 6.1.7 Immunofluorescence Staining

Paraffin sections (5 μm) of distal colons were dewaxed and rehydrated in degressive concentrations of ethanol. Endogenous peroxidase activity was blocked by 3% hydrogen peroxide. Antigen retrieval was performed with 10 mM sodium citrate buffer at pH 6.0 for 10 min in microwave, followed by blocking of non-immunological binding with 5% bovine serum albumin (BSA) for 2 hr. Sections were then incubated overnight at 4 □C with rabbit anti-claudin 1 antibody (1:100 dilution; ab15098, Abcam), After several washes with PBS, sections were incubated with Alexa Fluor 488 donkey anti-rabbit IgG (1:500 dilution; Invitrogen) [35]. Sections were counterstained with Hoechst 33258 (Invitrogen). Images were captured using confocal microscope.

### 6.1.8. In Situ Hybridization

Sense and antisense riboprobes for mouse Mrgprb2 were generated from their respective partial cDNA clone (503 bp) containing pBlueScript KS+using a digoxigenin RNA labeling Kit (Roche Diagnostics) by inserting the partial sequence between HindIII and XbaI restriction enzyme sites in pBlueScript KS+with Mrgprb2 forward primer CAGTCTAGAAAGCACCTCAGCCTGGAAAA and Mrgprb2 reverse primer CAGAAGCTTGTAAGCCACATGCCTTCCCT. Colon sections were rehydrated, treated with proteinase K, and then acetylated before incubation at 50° C. for 1 hr with prehybridization buffer (pH 7.5) containing 50% formamide, 0.6 M NaCl, 10 mM Tris-HCl, 1.3×Denhardt's solution, 1 mM EDTA, 50 μg/mL herring sperm DNA, and 50 μg/mL yeast tRNA. Hybridization was conducted during overnight incubation with heat denatured sense and anti-sense probes (0.25 ng/μL) at 50° C. in the same buffer supplemented with 10 mM DTT, 10% dextran sulfate. Post-hybridization treatment and incubation with anti-digoxigenin (1:400) antibody conjugated to alkaline phosphatase (Roche Diagnostics) and NBT/BCIP substrate was used to probe the signal. 0.1% fast green was used to counterstain the samples before imaging with Nikon Eclipse Ni-U upright microscope (Nikon H550L).

### 6.1.9. Cytokine Array Analysis

The cytokine membranes were blocked with blocking buffer in room temperature for 1 hr. The blocking buffer was removed from the membranes and 100 L of plasma obtained from the treatment groups (WT+Water, WT+2% DSS, Mrgprb2−/−+Water and Mrgprb2−/−+2% DSS; Water+Saline, 2.5% DSS+Saline, 2.5% DSS+P17*A7 and 2.5% DSS+GE1111) diluted in blocking buffer (total volume 1 mL) were subjected to membrane containing array of antibodies anti-cytokines. The membranes were first incubated at 4 □C for 24 hr in the blocking buffer, following five washes with washing buffers (I and II) and then incubated with 1× biotin-conjugated anti-cytokines overnight at 4□C. Membranes were washed again with washing buffers (I and II) 5 five times and incubated with 1×HRP-conjugated streptavidin for 2 hr at room temperature. Membranes were washed again, and 1 mL of detection buffer C and D were mixed and added to each membrane in dark for 2 min before imaging. Finally, array images were measured with ChemiDoc MP (Bio-Rad Laboratories, Hercules, CA). The detailed protocol can be referred to the instructions of a Cytokine Antibody Array Kit (ab133999—40 targets, Abcam).

6.1.10 Enzyme-linked immunosorbent assay (ELISA)

Concentrations of cytokines in blood plasma of the treatment groups (WT+Water, WT+2% DSS, Mrgprb2−/−+Water, Mrgprb2−/−+2% DSS, Water+Saline, 2.5% DSS+Saline, 2.5% DSS+P17, 2.5% DSS+P17*A4, 2.5% DSS+P17*A7, 2.5% DSS+CST-14 and 2.5% DSS+GE1111) were determined using ELISA kits (M-CSF-ab199084, Abcam; LIX—ab264611, Abcam; sTNF RI—ab202408, Abcam; G-CSF—ab197743, Abcam; BLC-EMCXCL13, Thermofisher) according to the manufacturer's protocol. Optical densities of the samples and standards were measured using PerkinElmer Victor X4. Cytokine concentrations were determined by comparing the optical density from the standard curve of each cytokine standards.

6.1.11. PRESTO-Tango GPCR P-arrestin recruitment assay

HTLA cells were maintained in DMEM supplemented with 10% FBS, 2 μg/mL puromycin and 100 μg/mL hygromycin B in a humidified atmosphere at 37° C. in 5% CO2. One million cells were seeded in 100 mm cell culture dish (Day 1). Cells were transfected with 4 μg Tango-MRGPRX2 construct using Lipofectamine 2000 reagent (Day 2). On day 3, cells were harvested and 20,000 cells per well were plated in 80 μL medium into poly-L-lysine coated and rinsed 96-well white cell culture plates (PerkinElmer). On day 4, GE1111 (final concentration 10 PM) is added 1 hr prior to the agonists or antagonist in dose response manner (Compound 48/80, P17, P17*A4, P17*A7 and GE1111). On day 5, medium and drugs were removed from the wells slowly (aspiration) and 30 L luciferase substrate solution (500 mM DTT, 10 mM CoA, 100 mM ATP, 80 mg/mL luciferin and triton lysis buffer) were added to each well. After incubation for 10 min at room temperature, luminescence was counted in PerkinElmer Victor X4. RLU (relative luminescence units) results were exported into Excel sheets and GraphPad Prism 10 was used for analysis of data. To measure constitutive basal activity, no ligand was added on day 4.

### 6.1.12 Flow Cytometry Assay

LAD2 cells (1×10⁶) were plated in 6-well tissue culture treated plates in 1 mL StemPro-34 culture media. Cells were treated with Compound 48/80 (10 μM), P17 (10 μM), P17*A4 (10 μM), P17*A7 (10 μM) and GE1111 (100 μM) for one hour. Cells were harvested and centrifuged at 500 μg for 5 min. Cell pellets were washed and resuspended in FACS buffer (0.05% sodium azide, 0.5 μg BSA in 100 mL PBS). Cells were resuspended in 400 μL FACS buffer and divided equally (100 μL) for isotype controls (total and surface) and MRGPRX2 staining (total and surface). 5 L of Human MRGPRX2 Alexa Fluor® 647-conjugated Antibody (Catalog #: IC4727R, R&D systems, USA) were added to the cells and incubated for 30 min in dark at 4° C. For isotype controls (FITC conjugated IgG2a) were added in cells. For total MRGPRX2 staining, cells were permeabilized with Cytofix/Cytoperm (BD Biosciences) for 30 min and washed with PBS before adding antibodies. Stained cells were washed with PBS for three times and resuspended in 500 μL FACS buffer. Cells were sorted using BD FACSAria™ III cell sorter. Analysis was done using FlowJo V10 software.

### 6.1.13. Western Blot

LAD2 cells were treated with agonists (1 μM Compound 48/80, P17, P17*A4, P17*A7 and 25 μM GE1111) for 15 min. For antagonist study, GE1111 (25 μM) were added 30 min prior to adding the agonists (1 M Compound 48/80, P17, P17*A4 and P17*A7). Cells were harvested and RIPA buffer containing cocktail mix of phosphatase and protease inhibitors was used to extract protein. Upon Bradford assay, harvested proteins were denatured with Lamelli buffer at 95° C. for 5 min. Protein samples were loaded in SDS-PAGE and transferred to nitrocellulose membrane. Membranes were blocked with 5% BSA for 1 hr and incubated at 4° C. overnight with primary antibodies (1:1000) in BSA (p44/42 MAPK (Erk1/2) (137F5) Rabbit mAb #4695, cell signalling, USA; Phospho-p44/42 MAPK (Erk1/2) (Thr202/Tyr204) (D13.14.4E) XP® Rabbit mAb #4370, Cell Signalling, USA; GAPDH (14C10) Rabbit mAb #2118, Cell signalling, USA; STIM1 Antibody #4916, Cell Signalling, USA) followed by (1:2000) HRP linked anti-rabbit secondary antibody (Anti-rabbit IgG, HRP-linked Antibody #7074) in 1 hr at room temperature. ECL was added and blots were visualized with Bio Rad CHEMIDOC™ MP imaging system.

### 6.1.14. Disease Activity Index Assessment

All animals were examined daily and the disease activity index (DAI) score was assessed as previously described [36] by assessing stool consistency, presence of blood in stool and body weight change as summarised in Table 1. Percentage of body weight was calculated relative to the initial body weight at Day 0 [(Weight on a Day−Initial weight at Day 0/Initial weight at Day 0)×100].

### 6.1.15. Quantification and Statistical Analysis

All data are shown as means±standard error mean (S. E.M) unless specified. The graphs between groups were plotted using Prism 8.0 software (GraphPad Software Inc, San Diego, CA). The data were analyzed using Student t-tests, 1-way ANOVA and 2-way ANOVA followed by Dunnett's multiple comparisons test throughout this study. A p value of less than or equal to 0.05 was significant.

### 6.2. Results

### 6.2.1 Knockout of Mrgprb2 Gene in Mice Exacerbates the Severity of Colitis in DSS-Induced Colitis Model

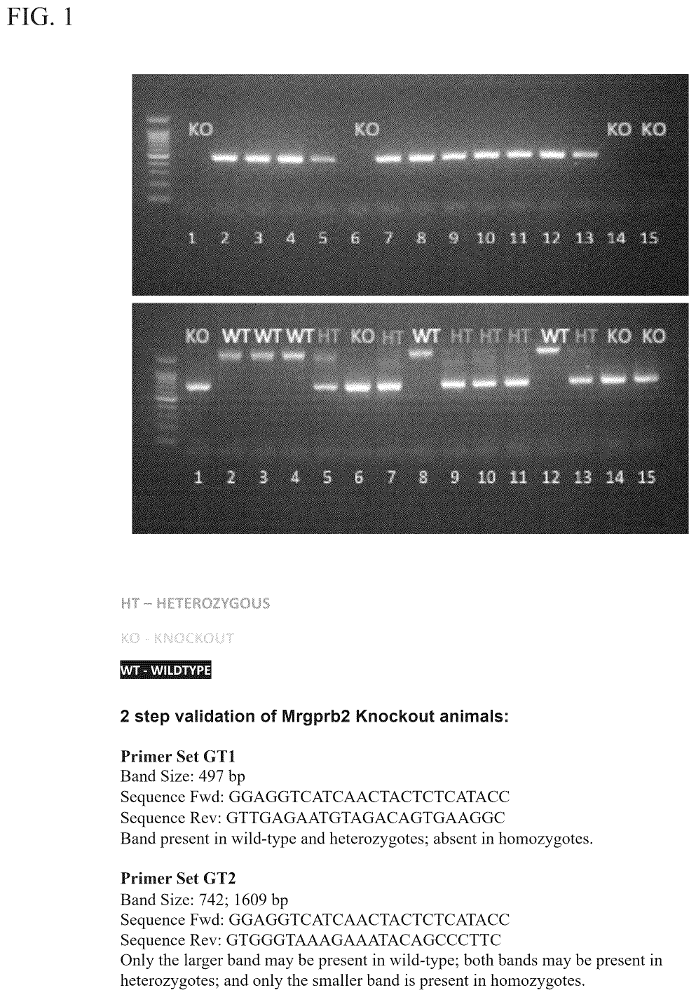

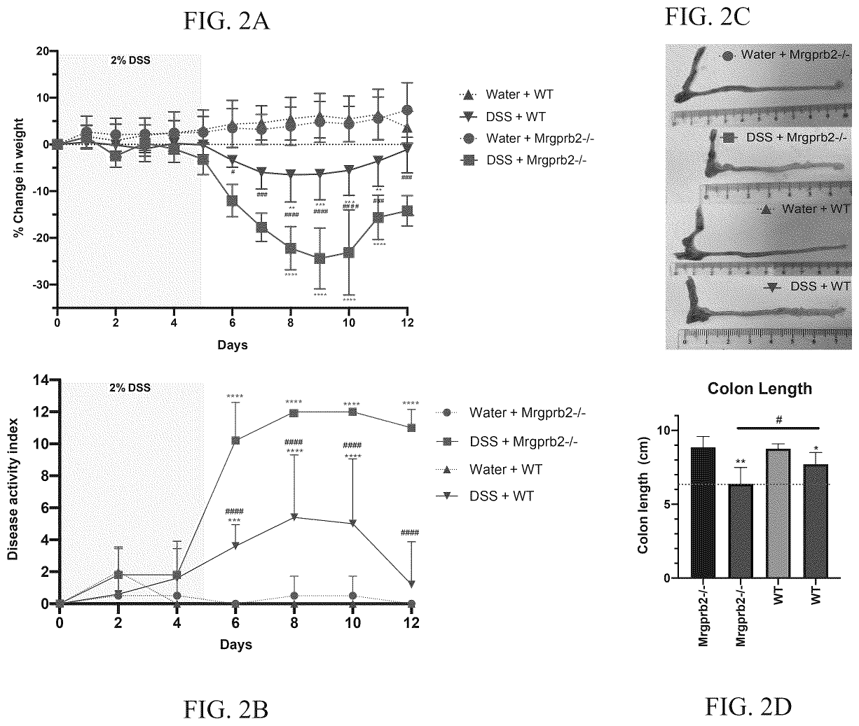

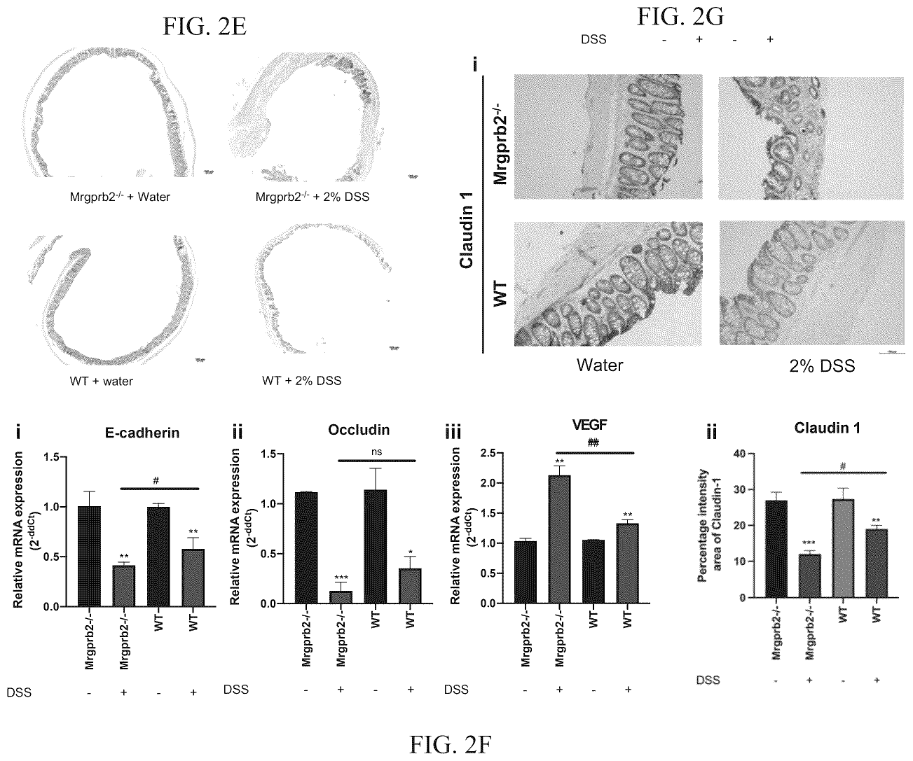

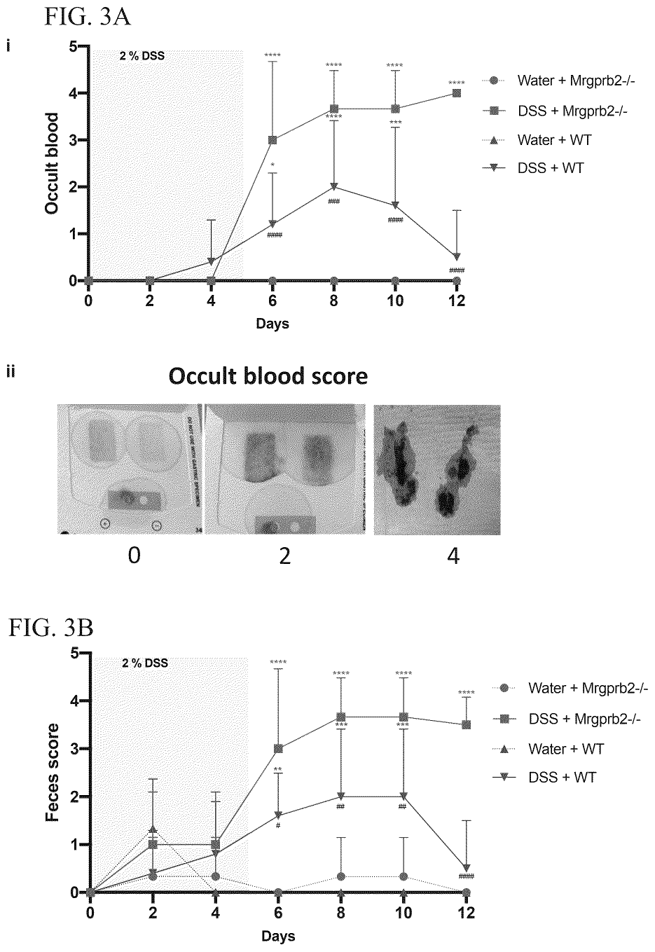

To understand the physiological role of Mrgprb2 in the pathology of colitis, Mrgprb2 knockout mice were used in DSS-induced colitis model along with C57BL/N WT animals. Mrgprb2 knockout validation was performed using genotyping (FIG. 1). Body weight of the Mrgprb2^{-/-} mice reduced significantly compared with the WT mice after 2% DSS treatment, while the bodyweight of the water treated control groups remained similar between Mrgprb2^{-/-} mice and WT mice (FIG. 2A). Disease activity index (DAI) score, which comprised of factors tabulated in Table 1 and FIG. 3, shows that scores increased significantly after 2% DSS intake in both Mrgprb2^{-/-} and WT groups while Mrgprb2^{-/-} mice had a higher DAI as compared to the WT mice group (FIG. 2B). Since colitis results in superficial inflammation characterized by epithelial erosion, ulceration, crypt abscess, loss of mucosal layers and substantial recruitment of inflammatory immune cells [37], colons were harvested to assess the inflammation and their length in 2% DSS-treated mice and control groups. From 2% DSS-treated experimental groups, Mrgprb2 knockout showed significant reduction in colon length as compared to the WT animals. However, from the water treated control groups, Mrgprb2^{-/-} and WT mice had no changes in colon length (FIG. 2C-D). In order to observe the colonic erosion and histological changes, H&E-staining was performed on colon tissues. H&E-stained colon tissues indicated that Mrgprb2^{-/-} mice had severe colonic epithelial erosion as compared to the WT mice from the 2% DSS group, whereas the water treated control group's mice had no significant changes (FIG. 2E). To go further, qRT-PCR analysis of genes associated with colon integrity such as E-cadherin, occludin were quantified. Data indicate that WT and Mrgprb2^{-/-} mice from 2% DSS group had significant reduction in the expression of the colon integrity genes as compared to the water treated control groups (FIG. 2F). Moreover, E-cadherin and occludin mRNA expressions were significantly reduced in Mrgprb2^{-/-} mice than the WT mice in 2% DSS group indicating the role of Mrgprb2 in colon integrity. On the other hand, VEGF transcript expression was significantly increased in Mrgprb2^{-/-} mice more than in WT mice from 2% DSS group as compared to the respective control group (FIG. 2Fiii). In addition, IHC images of colon tissues showed a significant reduction in the claudin-1 expression in Mrgprb2^{-/-} mice as compared to the WT mice from 2% DSS group. Collectively, our findings showed that Mrgprb2 receptor knockout aggravates the effect of DSS-induced colitis and indicate the significant protective role of Mrgprb2 in UC.

### 6.2.2 Knockout of Mrgprb2 Gene Results in Immune Cell Infiltration in Colon Through Altered Cytokine Releases

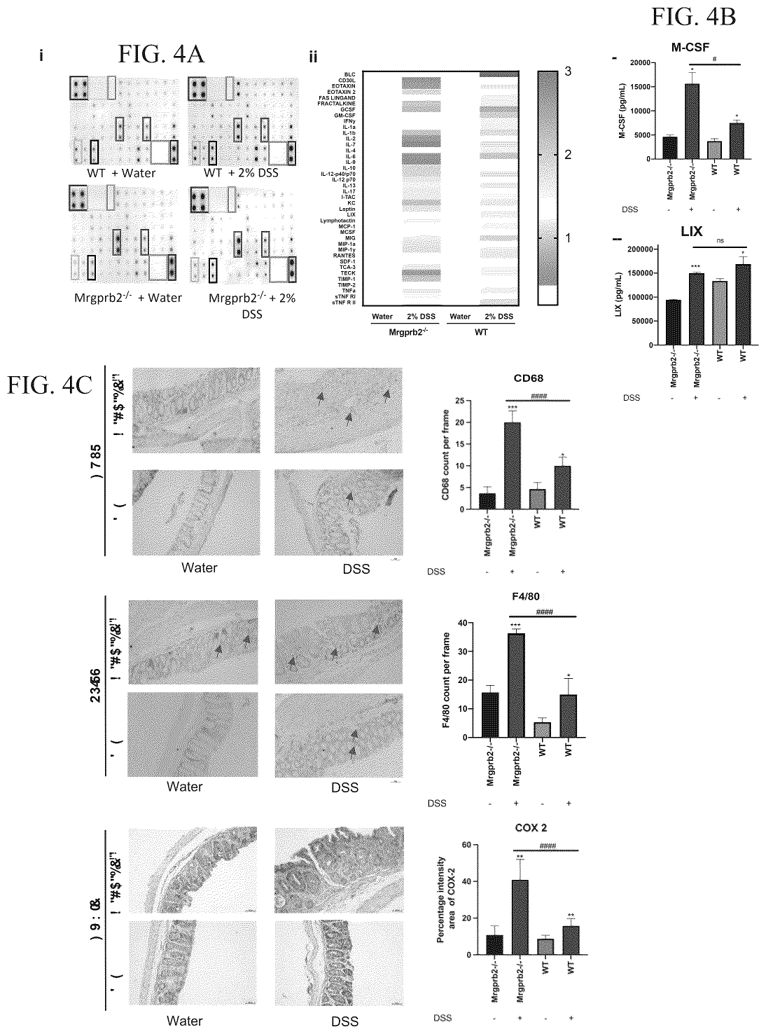

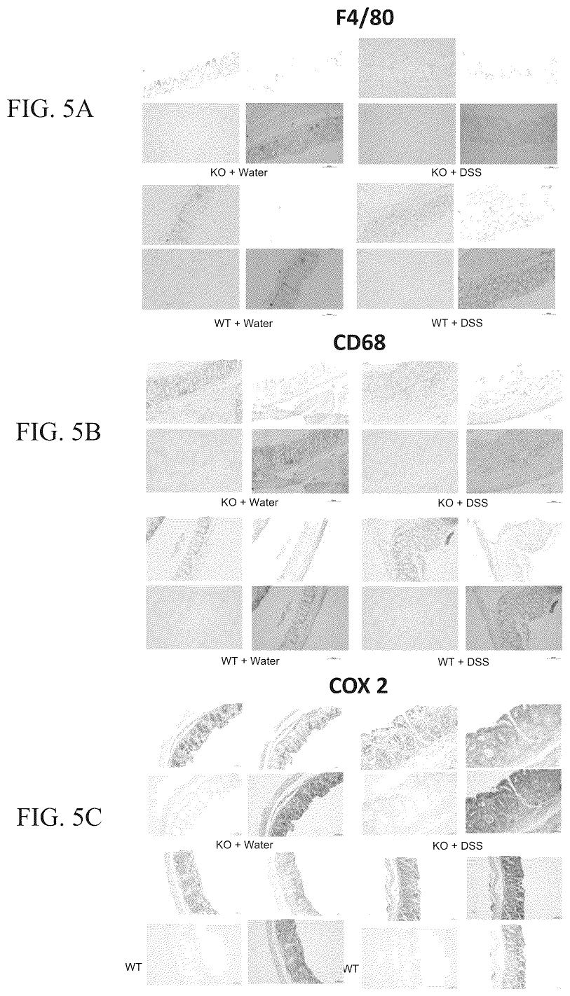

To further the understanding of the pathology of colitis in Mrgprb2^{-/-} mice, we screened for cytokine and chemokine releases in the blood plasma using cytokine membrane arrays consisting of 40 different capture antibodies. We exposed the membranes with the plasma samples harvested from the WT and Mrgprb2^{-/-} mice groups treated with and without 2% DSS (FIG. 4Ai). We listed the overall cytokine releases in terms of fold change between WT mice treated with water and 2% DSS, and Mrgprb2^{-/-} mice treated with water and 2% DSS, respectively. The graph shows the various cytokine levels that are differentially regulated between the control and the DSS treatment groups (FIG. 4Aii). Out of all the cytokines that are differentially regulated, we selected a few for ELISA assay to validate the data obtained from the cytokine array. For which, inflammatory markers M-CSF and LIX in the blood plasma of animals treated with 2% DSS were analysed with ELISA kits (FIG. 4Bi and 4Bii). Both WT and Mrgprb2^{-/-} mice showed elevated levels of M-CSF and LIX. Particularly, we found that there is a marked increase in M-CSF in Mrgprb2^{-/-} mice as compared to the WT mice treated with 2% DSS. Since there were altered cytokine releases in DSS-treated groups, in both WT and Mrgprb2^{-/-} mice, we evaluated the immune cell infiltration by measuring CD68 and macrophage marker F4/80 in the colons using immunohistochemistry (IHC). Moreover, in order to evaluate the inflammation in the colon, cyclooxygenase 2 (COX-2) was utilized since COX-2 expression is mainly induced at the sites of inflammation due to the inflammatory cytokine releases from inflamed cells, which is often absent in healthy cells [38, 39]. Our data showed that 2% DSS-treated Mrgprb2^{-/-} mice had significantly high infiltration of immune cells, inflammation, and damages in the colons than WT counterpart (FIG. 4C and FIG. 5). Taken as a whole, these results indicate that Mrgprb2 might play a crucial role in the pathology of UC as DSS treatment caused altered cytokine releases, which in turn leading to infiltration of immune cells and inflammation in Mrgprb2^{-/-} mice as compared to the WT mice.

### 6.2.3 Mrpgrb2/x2 Ligands Reduces the Severity of Colitis in DSS-Induced Colitis Animals

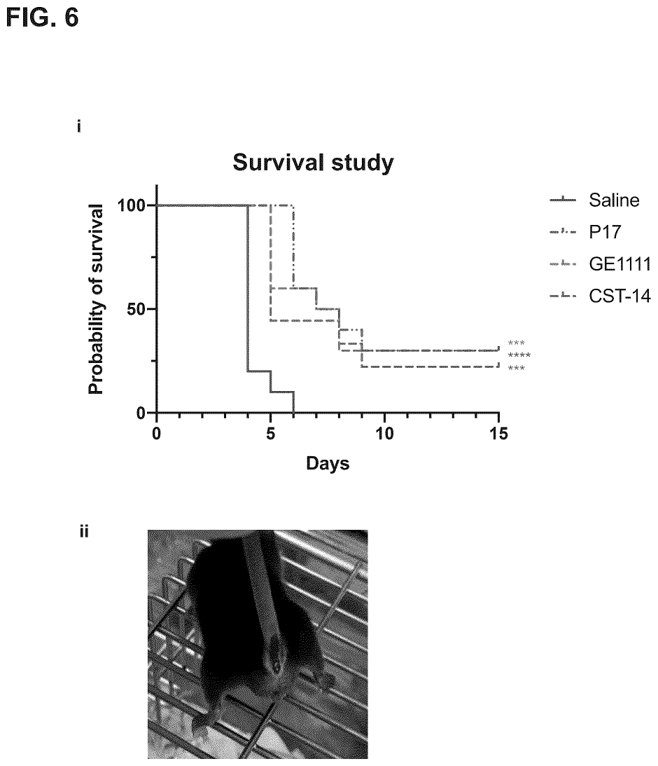

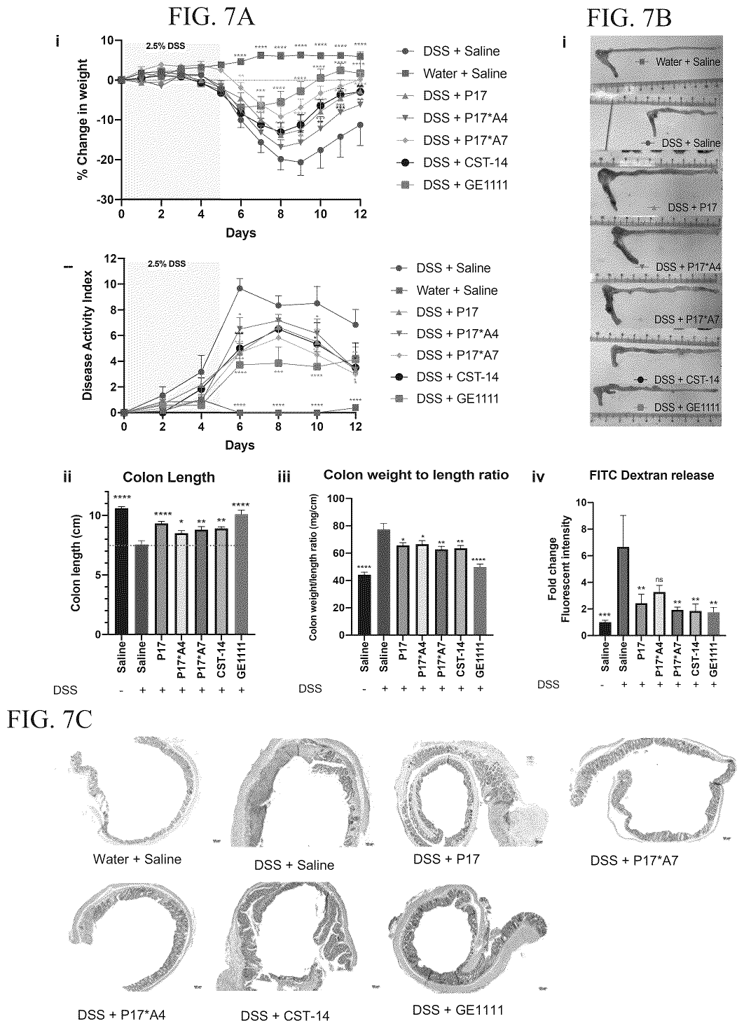

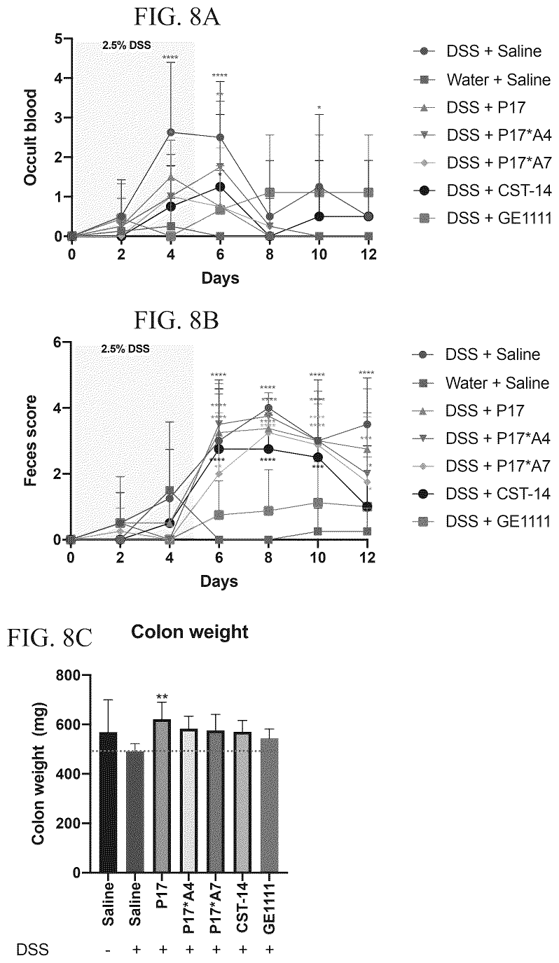

After showing a crucial role of Mrgprb2 in colitis model, we were interested in evaluating the effect of Mrgprb2/x2 modulation by its well-known agonists such as P17, Ala substituted P17 (P17*A4 and P17*A7) analogues, CST14 and its recently characterized antagonist GE1111 in DSS-induced colitis WT mice model. First, we performed survival study on mice treated with 4% DSS to observe the putative protective effect of Mrgprb2/x2 ligands. We showed that Mrgprx2 ligands, both agonists and the antagonist, protected the mice from the severe effect of 4% DSS (FIG. 6). Unexpectedly, GE1111 initially described as an antagonist of Mrgprb2 [25], mimicked the effect of the agonist on mice survival. We then reduced the dose to 2.5% DSS to understand the clinical parameters of colitis with Mrgprb2/x2 ligands. Body weight of the animals treated with 2.5% DSS showed a marked reduction as compared to the control groups or the treatment groups (P17 and P17 analogues, CST14 and GE1111). DAI scores were measured with factors such as body weight, occult blood, and faeces score (FIGS. 7A and 8). Scores were severe in 2.5% DSS treated group as compared to the control, and Mrgprb2/x2 ligands mitigated them (FIG. 7Ai and ii). Representative photographs displaying the excised colon from the experimental group show that DSS group had severe reduction in colon length compared to other groups (FIG. 7Bi-ii). Colon weight indicates the severity of the DSS effect as it would significantly reduce the colon length in colitis model. We showed that Mrgprb2/x2 ligands restored the weight loss of the colon (FIG. 8C). In addition, colon weight/length ratio was also calculated to understand the colonic severity due to weight gain caused by oedema or chronic inflammation. Indeed, DSS treatment affected the colon weight/length ratio significantly compared to the treatment groups (FIG. 7Biii). Moreover, mice treated with 2.5% DSS exhibited increased colon permeability for FITC-dextran whereas the treatments with Mrgprb2/x2 ligands reduced this permeability indicating Mrgprb2/x2 ligands protected intestinal barrier in this colitis model (FIG. 7Biv). To observe the colonic erosion and histological damages, we used H&E staining and found that 2.5% DSS treatment severely affected the colon and led to severe symptoms of colitis in saline injected groups compared to the Mrgprb2/x2 ligand injected groups (FIG. 7C). Treatments of both, Mrgprb2 agonists (P17, P17 analogues and CST-14) and antagonist GE1111 reduced the severity of DSS-induced colitis in WT mice.

### 6.2.4 Mrgprb2/x2 Ligands Treatment Reduces the Expression of Mouse Orthologue Mrgprx2 Genes in Colitis Models

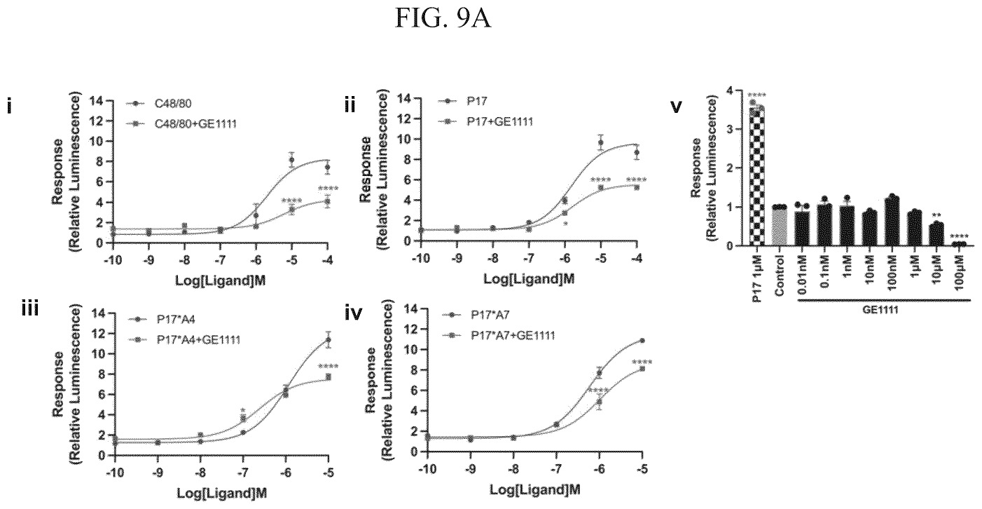

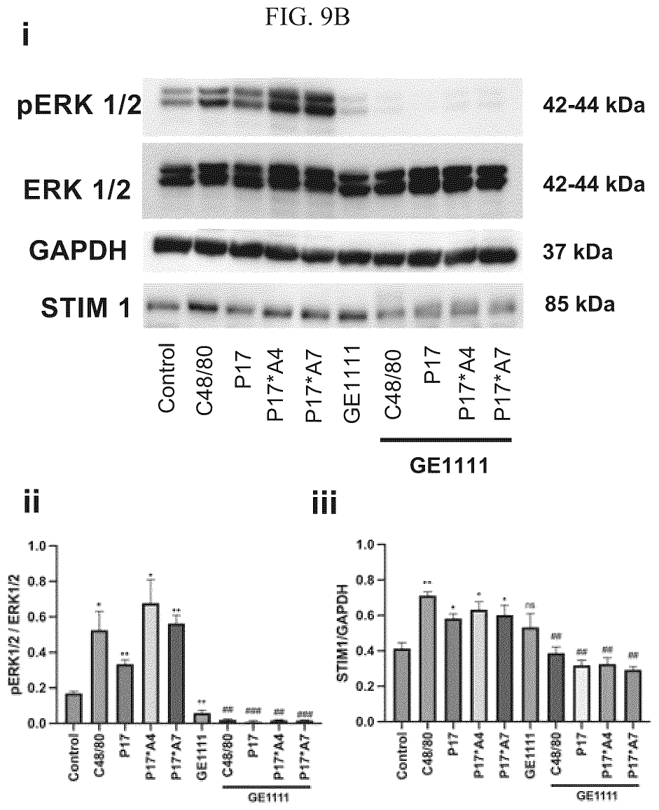

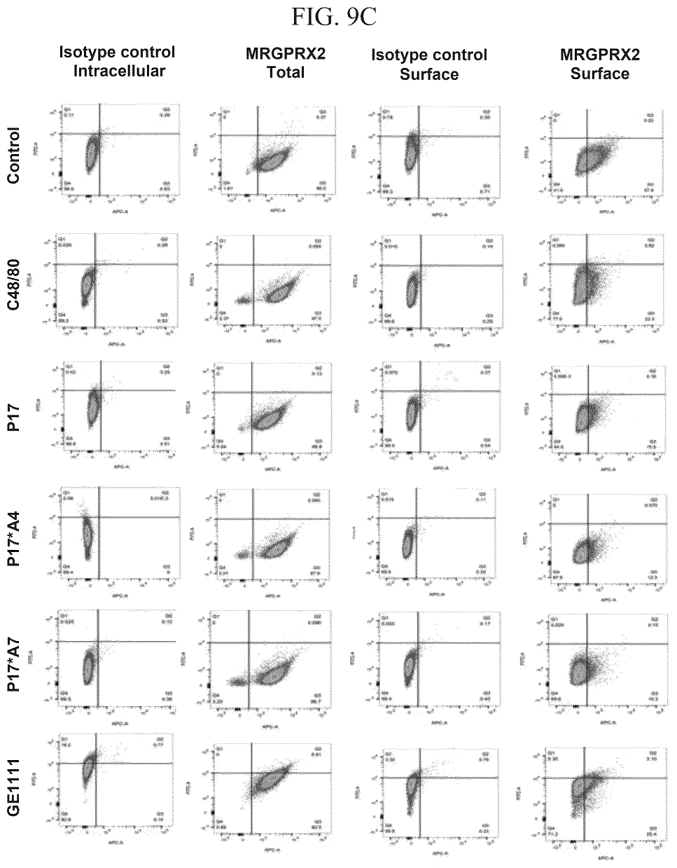

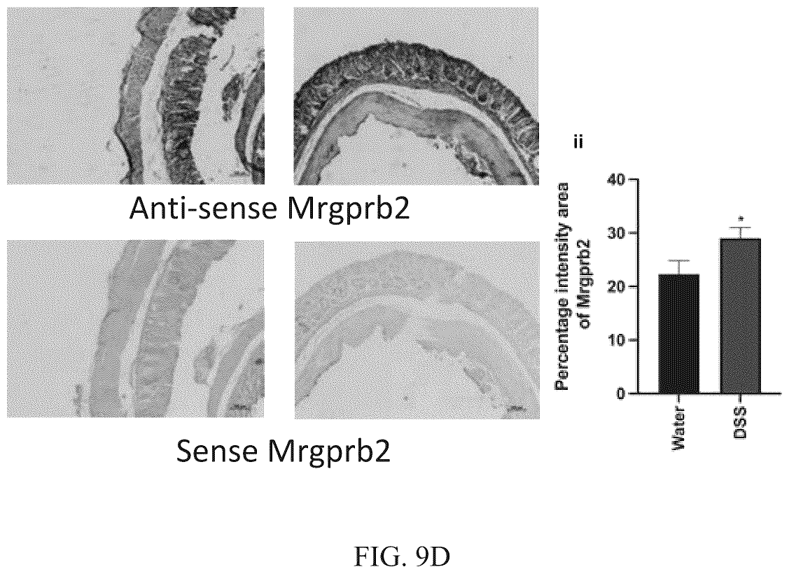

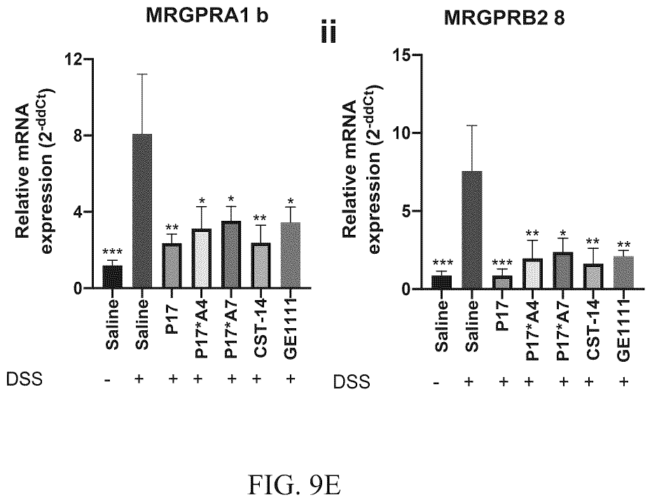

Since Mrgprx2 ligands, agonists and antagonist were shown to be protective in DSS colitis model. We first tested the effectiveness of the ligands on Mrgprx2 activation using Tango-GPCR b-arrestin recruitment assay. Compound 48/80, P17, P17*A4 and P17*A7 showed agonist activity and GE1111 antagonized the agonist activity (FIG. 9Ai-iv). Moreover, GE1111 dose response showed that GE1111 behaved as an inverse agonist (FIG. 4Av). Besides, western blot studies on LAD2 mast cells showed that the agonists treatment led to pERK 1/2 MAPK pathway and subsequent overexpression of stromal interaction molecule 1 (STIM1) that are crucial for store-operated Ca²′ entry in mast cells upon Mrgprx2 activation [40] and GE1111 antagonized this pathway (FIG. 9B). Moreover, flow cytometry analysis showed that Mrgprx2 ligands equally internalized Mrgprx2 upon treatment (FIG. 9C). It is to be noted that, Mrgprx2 expression has been shown to increase during UC in human colon [10], hence Mrgprx2 has been hypothesized to be a gene that promotes colitis development or severity of colitis. We tested the expression of Mrgprb2 in colon using in situ hybridization probes and we found that DSS treatment indeed increased the expression of Mrgprb2 in colitis models (FIG. 9D). Since, in vitro studies showed that Mrgprx2 ligands internalized Mrgprx2 upon treatment, we then investigated the effect of Mrgprb2/x2 ligands on the expressions of mouse orthologs of human Mrgprx2 in colon using quantitative RT-PCR. Expression of Mrgpra1 and Mrgprb2 were increased during DSS-induced colitis and the treatments with Mrgprb2/x2 agonists and GE1111 restored their expression to the levels of healthy mice (FIG. 9Ei and ii). These results suggests that elevated levels of Mrgprx2 might play a role in progression of colitis and targeting Mrgprx2 might be a viable option for the treatment of colitis.

### 6.2.5 Mrgprb2/x2 Ligands Restore Colon Integrity Genes in DSS-Induced Colitis Animals

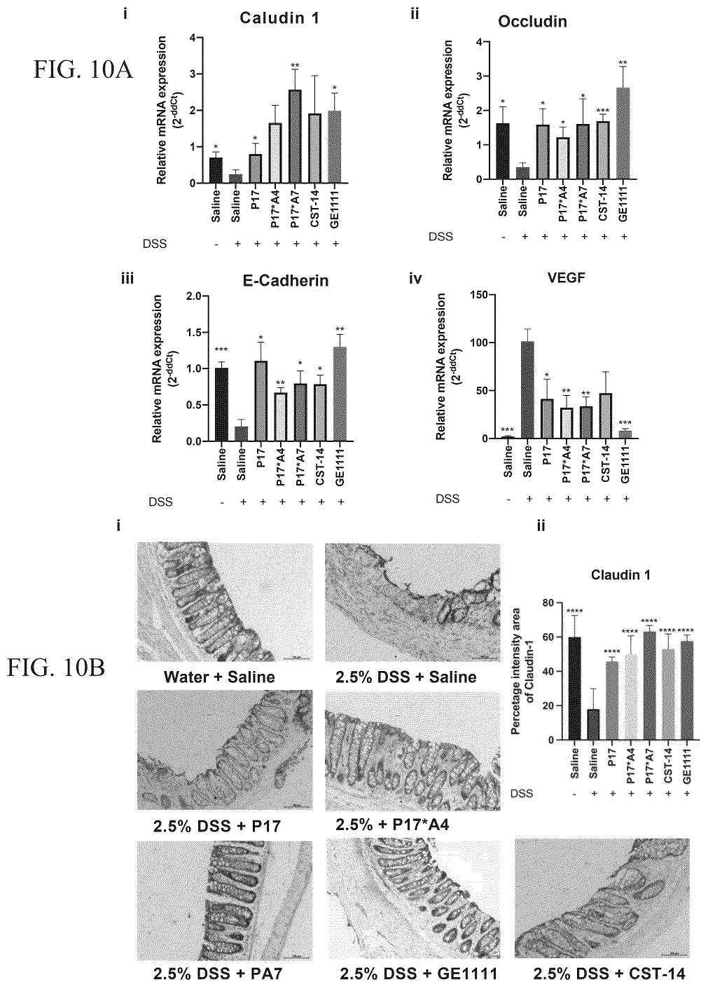

Progression of colitis leads to severe colonic erosion and epithelial damages; hence we have quantified the expression of major colon integrity genes such as claudin 1, occludin and E-cadherin as well as VEGF in WT animal treated with 2.5% DSS. We observed that the major junction proteins claudin 1, occludin and E-cadherin were significantly reduced in the DSS group without any treatments, whereas Mrgprb2/x2 agonists and GE1111 treatment were able to restore these gene expressions (FIG. 10Ai-iii). In addition, VEGF expression was significantly increased in the DSS group without any treatment and Mrgprb2/x2 ligands reduced the VEGF expression in DSS-treated mice (FIG. 10Aiv) indicating the protective effect of these ligands in DSS-induced colitis. Finally, we performed IHC on the colons with claudin 1 antibody and the expression of this protein was significantly reduced in DSS group without any treatment and restored in the presence of Mrgprb2/x2 ligands (FIG. 10Bi and ii). Moreover, immunofluorescence study with claudin 1 also produced the same results in the colon as IHC (FIG. 11).

### 6.2.6 Mrgprb2/x2 Ligands Alter Inflammatory Cytokines in Circulation and Attenuate Inflammatory Immune Cell Infiltration in Colon of the DSS-Induced Colitis Animals

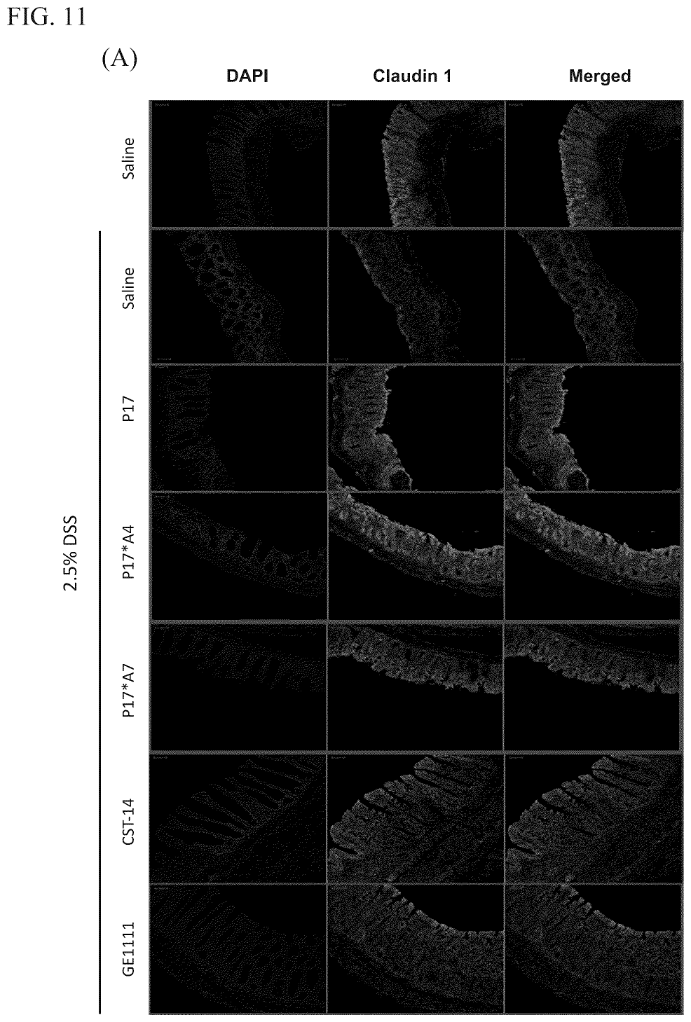

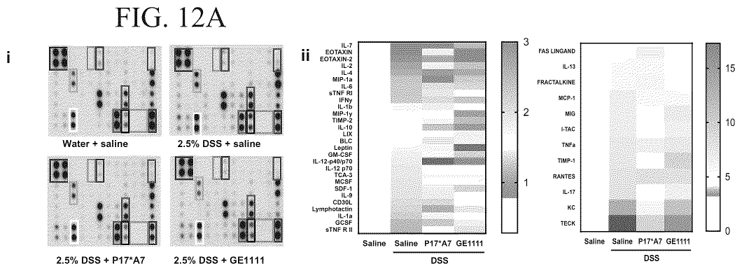

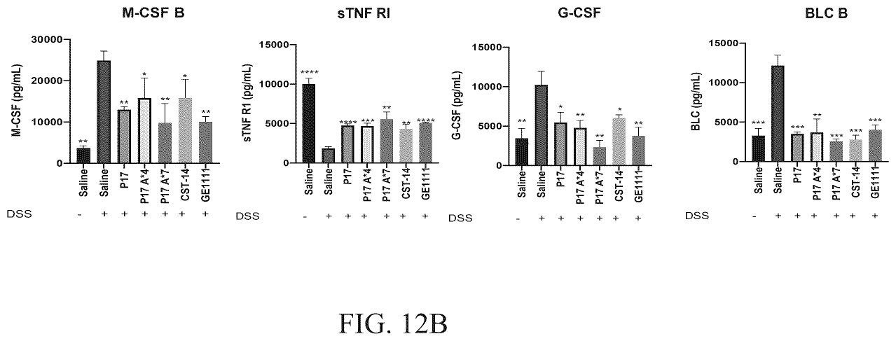

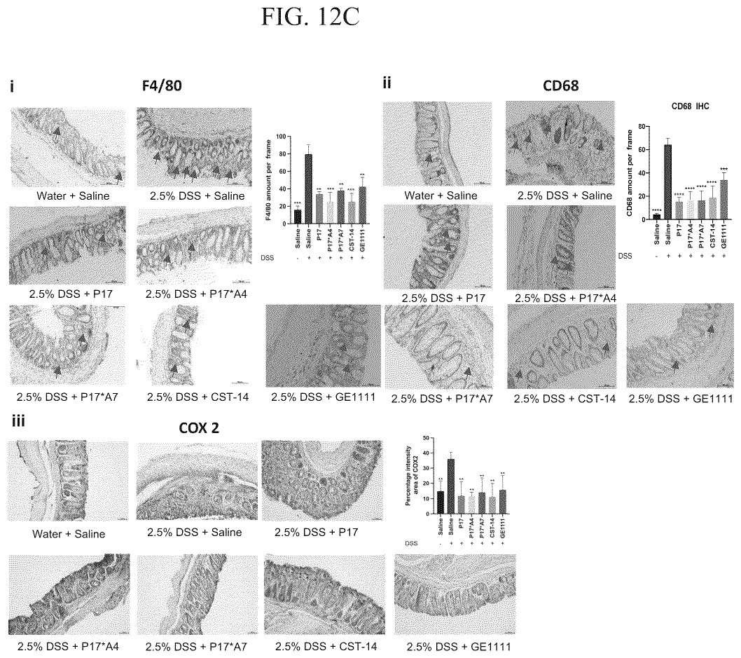

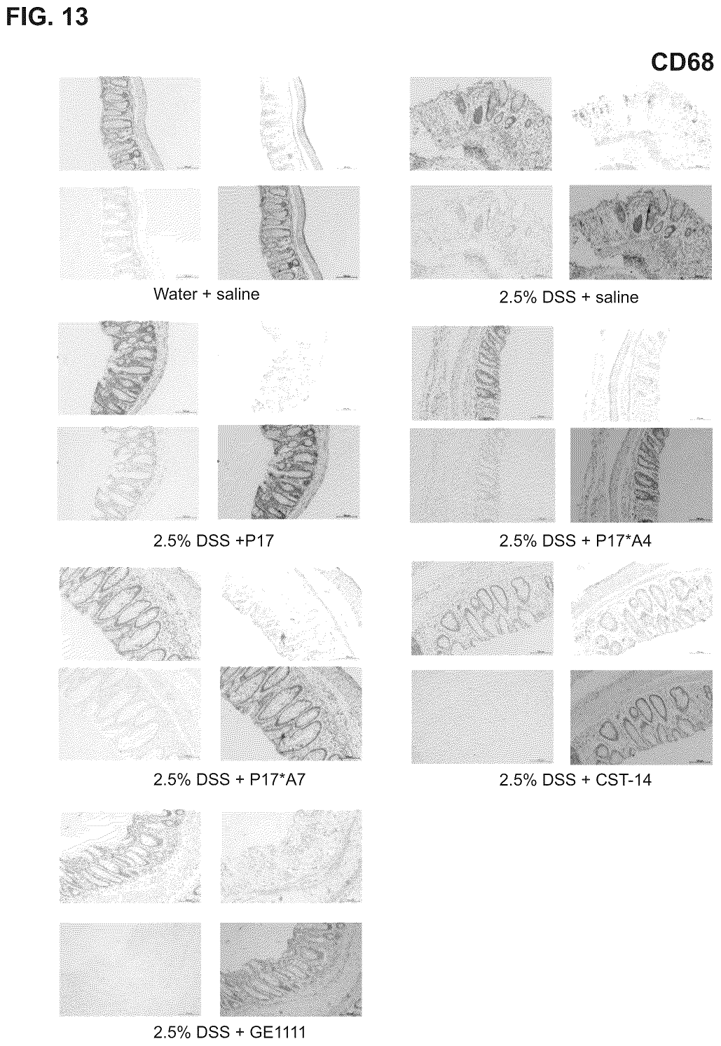

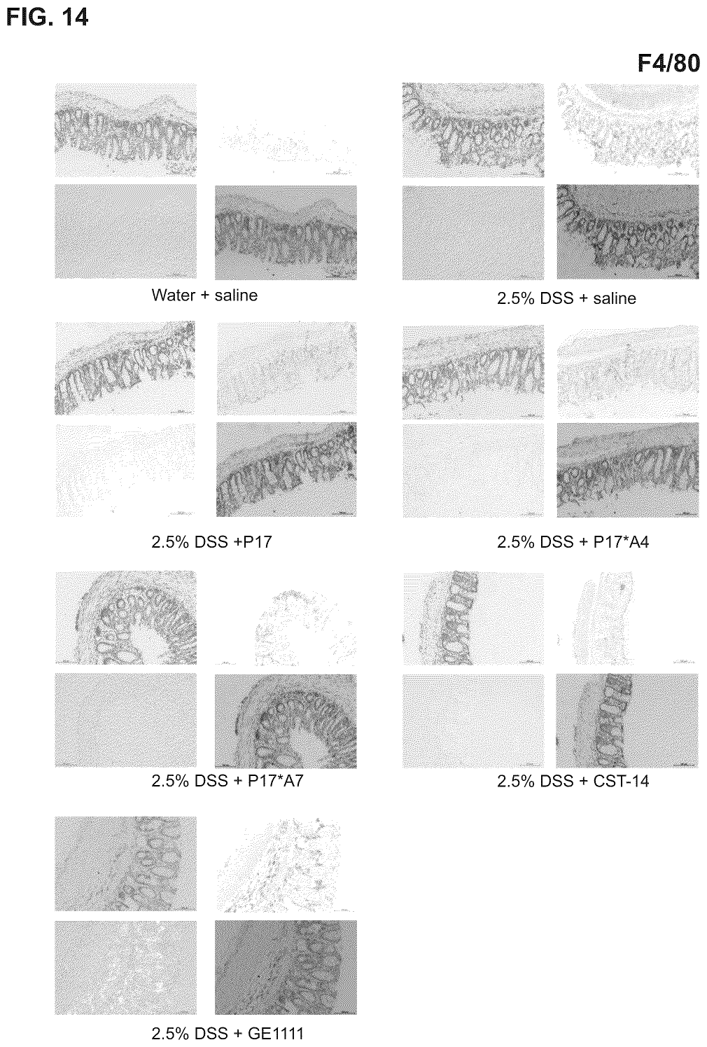

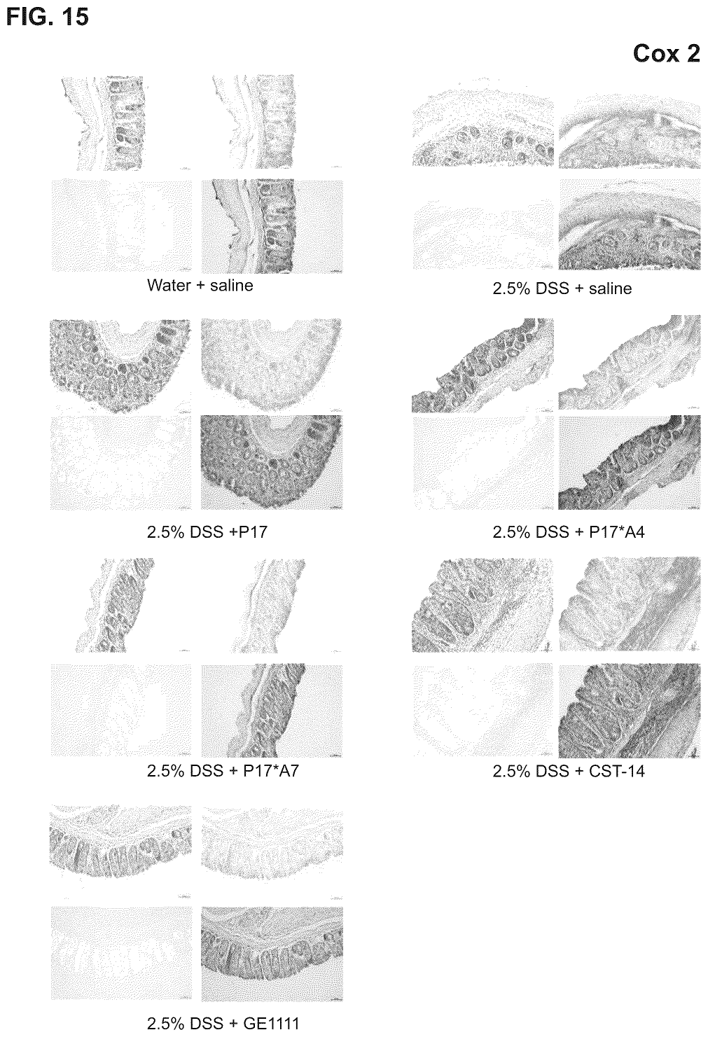

We screened for cytokine and chemokine releases in the blood of 2.5% DSS-induced colitis model in the presence or absence of P17*A7 and GE111 as described above (FIG. 12Ai). The group exposed with 2.5% DSS showed altered cytokine releases as compared to the water exposed group. In contrast, P17*A7 and GE1111 treatment administered to the mice exposed to 2.5% DSS led to significant modifications in most of the cytokine and chemokine levels in the blood plasma (FIG. 12Aii). In particular, P17*A7 and GE1111 treatment restored of some cytokines and chemokines to near baseline or maintained a significant reduction or increase compared to the saline injected mice exposed to 2.5% DSS (FIG. 12Ai-ii). Representative graphs of M-CSF, sTNF RI, BLC and G-CSF were analysed between all the groups using ELISA kits. M-CSF, BLC and G-CSF were elevated in mice group exposed to 2.5% DSS compared to the water exposed groups. In contrast, Mrgprb2/x2 agonists and GE1111 treatment led to a significant reduction in these cytokine levels (FIG. 12B). On the other hand, sTNF RI was significantly reduced in the saline injected mice group exposed to 2.5% DSS and Mrgprb2/x2 ligands partially maintained the plasma levels of sTNF RI (FIG. 12B). We analysed the immune cell infiltrations in colon using IHC for F4/80, CD68 and COX-2 markers. Saline injected mice group subjected 2.5% DSS showed increased immune cell infiltration and mice treated by Mrgprb2/x2 ligands partially attenuated the CD68 and F4/80 immune cell population in colon and COX-2 expression, respectively (FIG. 12Ci-iii). These infiltrations of immune cells and COX-2 markers were analysed using ImageJ software (FIG. 13-15). These results indicate that Mrgprb2/x2 agonists P17, P17 analogues, CST-14, and GE1111 analogously protect the mice from DSS-induced colitis.

### 6.3. Discussion

MCs and its specific receptor Mrgprb2/x2 promote wide range of immune and inflammatory responses. Particularly, in colon, they are prevalently found in the mucosae and clustered at the epithelial layer [10]. In addition, some of the endogenous peptides that activate Mrgprx2 are strongly expressed in UCs and DSS-induced colitis models such as PAMP-12 and LL-37 [10, 30]. Moreover, recent studies have shown that Mrgprx2 agonists such as protegrin [41] and CST-14 [42] as well as the antagonist quercetin [43, 44] ameliorate DSS-induced colitis symptoms in mice models. In our previous study, we demonstrated that P17, an ant venom peptide isolated from Tetramorium bicarinatum exhibits antimicrobial effects when the gastrointestinal track is infected with Candida albicans through activation of an unknown GPCR [45] and we subsequently characterized it as Mrgprb2/x2 [22]. Moreover, upon finding various cytokines being released by human MCs activated with P17 showed key cytokines that may be involved in colon integrity, such as the macrophage migration inhibitory factor, essential for intestinal barrier, and the neutrophil-activating protein 2, paramount for recruiting mesenchymal stem cells for tissue repair and wound healing. Hence, we hypothesized that Mrgprx2 and MCs may have potential role in inflammatory bowel diseases such as UCs [22]. On the other hand, we have also developed a non-peptide antagonist of Mrgprx2 that protects mice from anaphylactoid allergic reaction [25]. Since, the agonist and the antagonist of Mrgprb2 has shown to modulate different immunity or immune related disorders, we decided to explore the potential role of this AMP, P17 and its analogues (P17*A4 and P17*A7), and the antagonist (GE1111) in DSS induced colitis model.

As we proposed Mrgprb2 and its ligands in inflammatory bowel diseases [22], we sought out to evaluate the severity of colitis in DSS-induced colitis in Mrgprb2^{-/-} mice in comparison to WT animals. Herein, we show that Mrgprb2 knockout in colitis mice model, aggravated the effect of DSS and led to severe colonic damages by altering the expression of tight junction proteins claudin 1 and occludin and the adherent protein E-cadherin. Moreover, Mrgprb2^{-/-} mice showed elevated expression of VEGF as compared to the WT mice in colon objectifying the severity of the colitis and the angiogenesis of new blood vessels necessary for wound healing and tissue repair in UCs [46, 47]. Dysregulated and differential regulations of cytokines in Mrgprb2^{-/-} mice exposed to DSS show that Mrgprb2 is eminent for modulating the cytokines during the pathogenesis of the UC. Also, altered cytokine releases in the Mrgprb2^{-/-} compared to WT mice exposed to DSS mice indicating the regulatory role of Mrgprb2 in cytokine releases during UCs. For instance, pro-inflammatory cytokines M-CSF was significantly elevated in the knockout mice than in the WT mice. Elevated M-CSF expression is associated with M-CSF dependent macrophage recruitment to produce pro-inflammatory effect in colitis [48, 49]. Macrophages are classified into classically activated, pro inflammatory (M1) or alternatively activated, anti-inflammatory (M2) macrophages [50]. It is to be noted that, recruited macrophages can be either M1 or M2. In our previous study, we demonstrated that activation of Mrgprx2 in mast cells leads to recruitment of monocytes and their differentiation into M1-like phenotype [22]. Herein, we demonstrated that dysregulated release of cytokines subsequently recruited more F4/80⁺ and CD68⁺ marker immune cells at the colon possibly that are pro-inflammatory. Moreover, inducible inflammatory marker COX-2 expression in colon showed the severity of colitis in Mrgprb2^{-/-} mice. Indeed, these findings of Mrgprb2 in colitis is surprising as Mrgprb2 and its activation are often associated with severity and pathogenesis of UC [10, 19] and allergic diseases [51]. Thus, ideally, knockout of Mrgprb2 should rather result in reduced inflammation and reduced severity of UCs. Herein, our data suggests the opposite as Mrgprb2 knockout in mice aggravates the inflammation in DSS-induced colitis instead of mitigating it. A recent study confirms that Mrgprb2 gene invalidation exacerbate colitis through altered immune function and intestinal microbiota [31]. Hence, the contrasting findings highlight the research gap that requires further attention. In this context, our data show elevated expression of Mrgprx2 mouse orthologs of Mrgprb2 and Mrgpra1 in colon in WT groups exposed to DSS. Thus, it is plausible to corelate that elevated Mrgprb2 expression means severe inflammation in colitis. However, our receptor knockout findings contradict this claim since lack of Mrgprx2 results in severe inflammation. It is thus possible that basal level or presence of Mrgprb2 expression is important for the protective effect in colitis. Therefore, in pathological situation, the organism may rely on Mrgprb2 expression to counteract the inflammatory responses. Consequently, other studies have linked increased Mrgprb2 expression as indicative of disease progression. Nevertheless, the elevated Mrgprb2 expression leads to increased constitutive activity that may play a role in colitis progression over time. Therefore, it is likely that maintaining a basal level of Mrgprb2 expression in WT mice is essential to counteract the effects of ulcerative colitis; and uncontrolled expression with heightened constitutive activity of Mrgprb2 may lead to severe colitis. In this scenario, the administration of Mrgprb2 ligands lead to a decrease in overall Mrgprb2 expression, resulting in a reduction of colitis pathology.

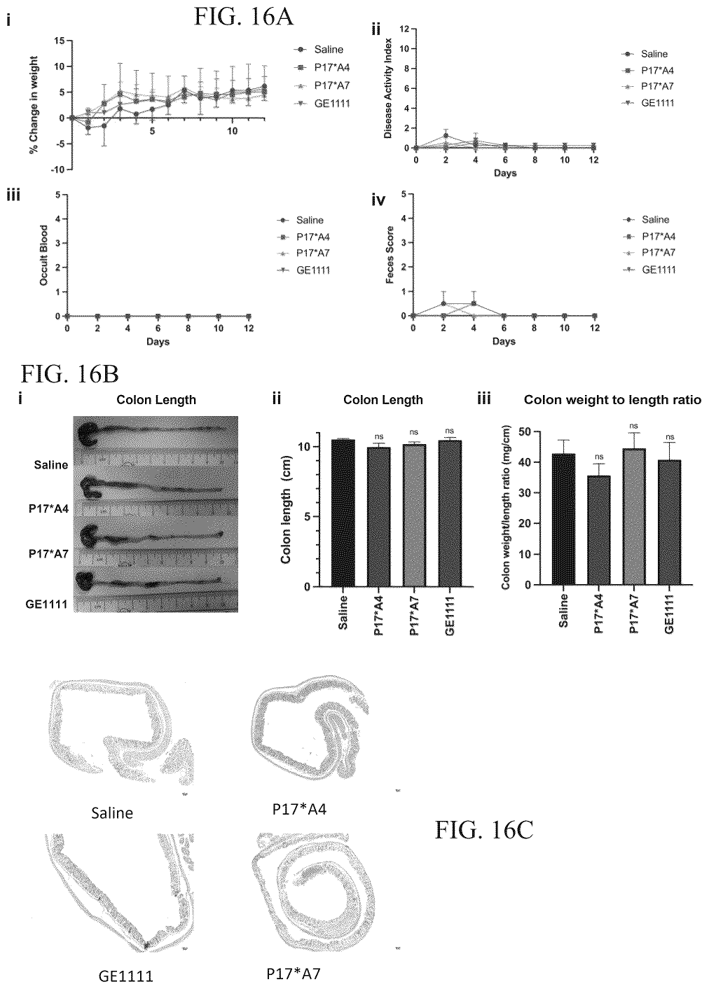

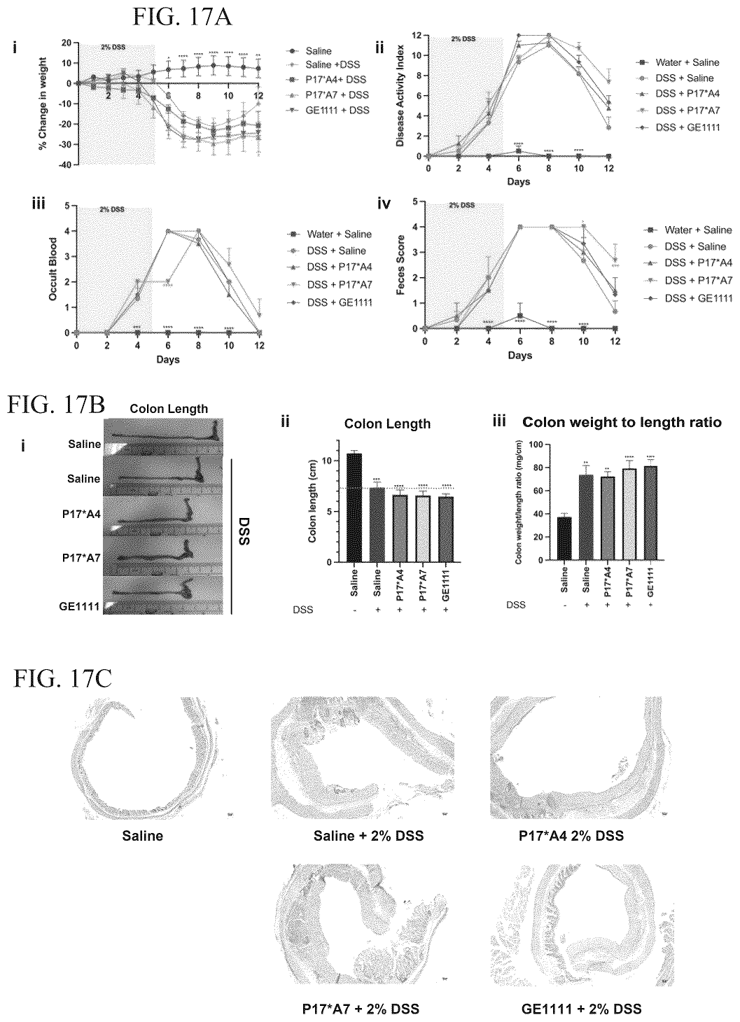

In this study, we demonstrated that Mrgprb2/x2 agonists and GE1111, initially reported as an antagonist analogously mitigated the severity of colitis. These results, although undoubtedly unexpected, prompted us to delve deeper into the underlying mechanisms involved in colitis to obtain a more comprehensive understanding of the observed phenomena. Both, agonists and GE1111 effectively reduced the expression of Mrgpra1 and Mrgprb2 in WT mice exposed to DSS. In turn, altering the cytokine releases as well as shifting the colon microenvironment towards anti-inflammatory environment to reduce the recruitment of inflammatory immune cells. For instance, sTNF RI is reduced in the DSS-treated animals indicating the severity of colitis as sTNF RI knockout in animals showed severe effects [52, 53], whereas Mrpgrb2/x2 ligands restored sTNF RI. Subsequently, Mrgprb2/x2 ligands resulted in less immune cell infiltration in the colon. In addition, Mrgprb2/x2 ligands protected the mice from severe intestinal barrier damage, demonstrated by the FITC-Dextran assay. In this context, the precise pharmacological profile of Mrgprb2/x2 ligands should be carefully considered. Our data showed that Mrgprb2/x2 ligands had no significant effect on the colon morphology in the absence of DSS in WT animals (FIG. 16). On the other hand, Mrgprx2 ligands were ineffective in Mrgprb2 KO mice treated with DSS (FIG. 17) indicating the protective effect is specific to Mrgprb2. Therefore, in this disease model, the protective phenotype of Mrgprb2/x2 ligands might be secondary to their known Mrgprx2 traditional signalling. While this study offers valuable insights into molecular and animal model aspects of UC pathology, there are more works to be done for the translatability of these findings to human UC treatment. Even though, sub-acute study of Mrgprb2/x2 ligands had no effect on mice (FIG. 16), but thorough toxicological studies will be carried out in near future before translating these findings to the clinical settings of UC in patients.

GPCR activation is not considered a linier pathway, since a single GPCR can lead to multiple intracellular biased signalling pathways depending on their ligands and the microenvironment [54-58]. Inverse agonism, antagonism, allosteric modulation, constitutive activity, oligomerisation and biased signalling are common for various GPCRs, hence the ligands cannot simply be classified as an agonist or an antagonist due to their complex mode of signalling pathways [57-62]. In addition, GPCR agonism often leads to receptor internalisation, desensitisation and recycling through GPCR kinases (GRKs) [63], which in turn will reduces the overall expression over a prolonged activation [64]e.g. β2-adrenergic receptors (β2ARs) and CXC motif chemokine receptor 4 (CXCR4) [65]. These receptors go through lysosomal degradation and resynthesis [65-67] hence it takes hours and sometimes days to reappear on the cell surface [64]. Activated Mrgprb2 receptors are known to be internalized by GRKs and recycled [63]. Conversely, GPCR antagonism can also result in receptor internalisation. For instance, Mrgprx2 expression were reduced in mast cells with osthole, a known inhibitor of Mrgprx2 [68] and Neuropeptide Y receptor Y1 is known to be internalized with its high affinity antagonist GR231118 and the internalized receptors were not recycled for hours [69]. Similarly, CCK1R is internalized upon its antagonist, d-Trp-OPE binding [70]. Overall, GPCR internalisation and degradation leads to reduced abundance of the receptor in the cell membrane. Moreover, agonist and antagonist may act as inverse agonists in certain environment. There are studies demonstrating either the antagonists switch to agonists [71] or agonists switch to antagonists [72, 73] at certain conditions or upon slight modifications. In addition, most of the common β-adrenergic receptor antagonists are known to act as inverse agonists in the absence of agonists [74, 75]. Most of the antihistaminic of the histamine receptors behave as inverse agonists [76]. In this study, through in vitro studies, we revealed that GE1111 acts as an inverse agonist and the Mrgprx2 ligands (agonists and antagonist) can equally internalize Mrgprx2. However, this study was limited in directly assessing the Mrgprb2 expression at the protein level in colon following ligand treatment due the unavailability of specific antibodies targeting Mrgprb2. Instead, qPCR studies were employed to show the indirect reduction of Mrgprb2 in colon. Nevertheless, our data shows elevated expression of Mrgprb2, meaning elevated constitutive activity. Therefore, it is plausible that both the agonist P17 and the antagonist GE1111 may result in degradation of the receptor following internalisation or act as inverse agonists to reduce the overall receptor activity in disease condition to reduce immune cell infiltration and inflammation. However, this needs to be studied in detail with different molecular approaches.

### 6.4. Conclusion

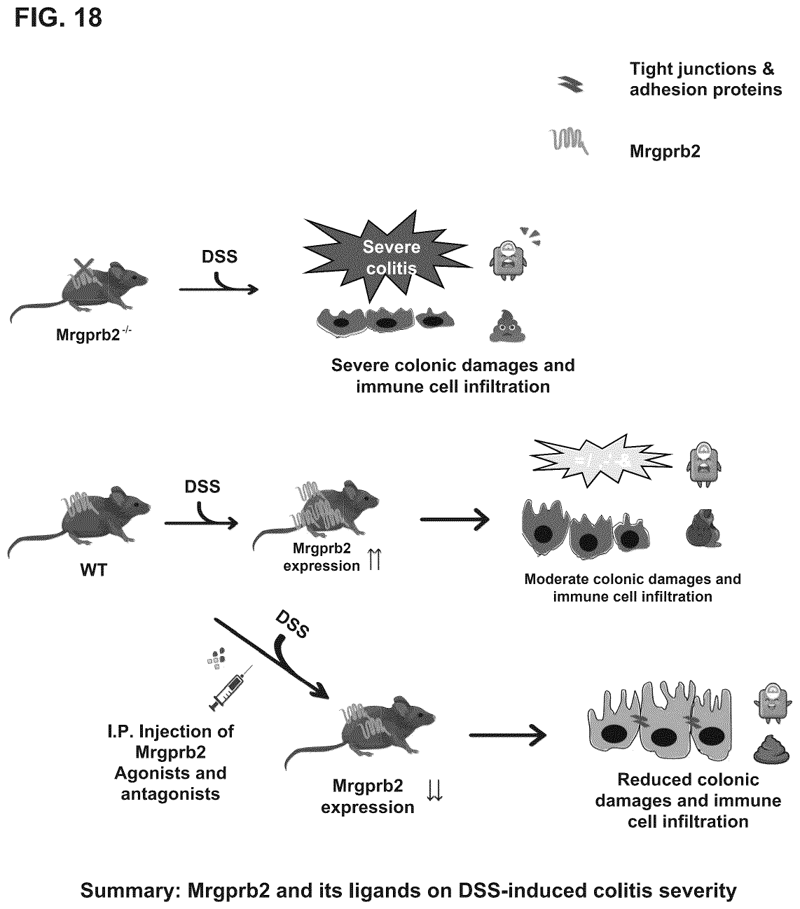

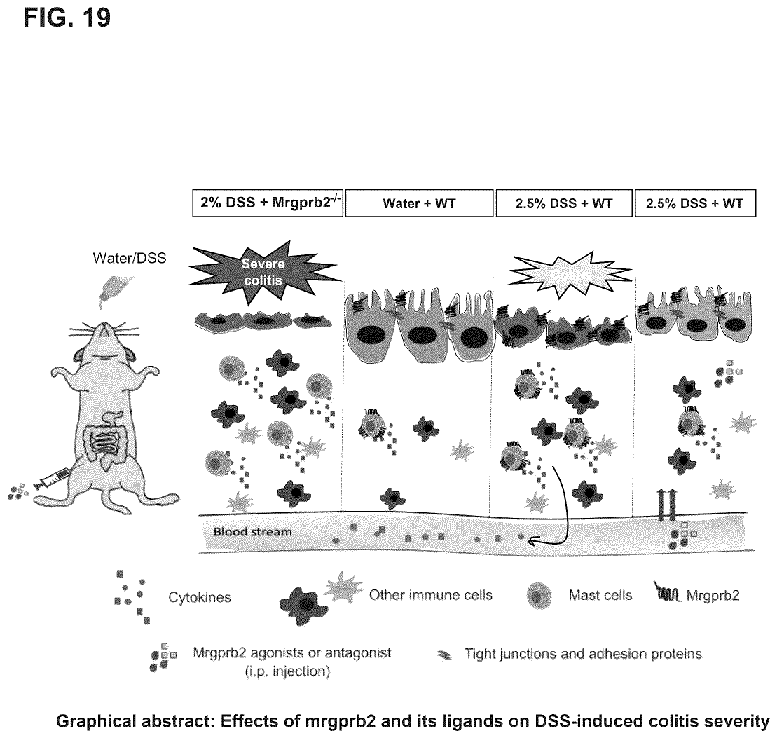

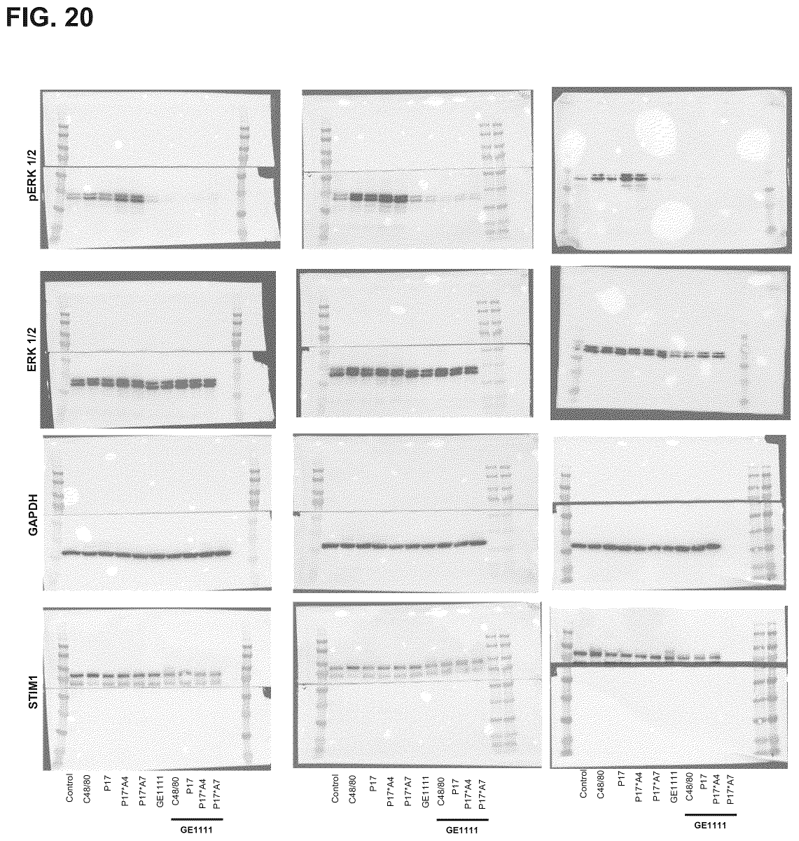

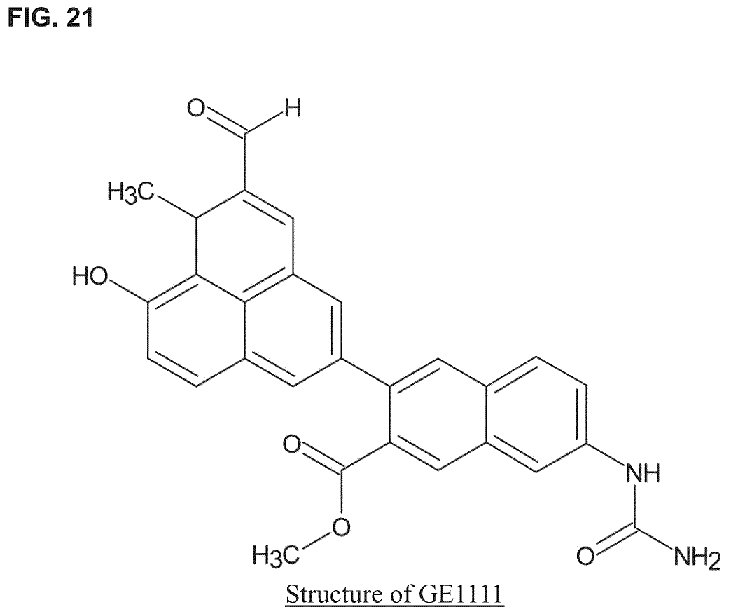

In summary, absence of Mrpgrb2 exacerbates colitis severity, and its overexpression in the disease model may represent a compensatory response by the organism. This observed duality of Mrgprb2 indicates that the molecular pathways involved in this context are a complex regulatory mechanism. To reiterate, our data demonstrates that Mrgprb2 is playing a protective immune regulatory role in DSS-induced colitis by altering cytokine releases and immune cell infiltration. In the absence of Mrgprb2, the usual functions of Mrgprb2 are disrupted leading to significant elevation of immune cell infiltration and loss of colon integrity proteins in mucosal epithelial layers causing UCs. Moreover, Mrgprb2/x2 ligands analogously reduce the elevated expression of Mrgprb2 in disease condition in WT animals, hence in turn reduce altered cytokine releases, immune cell infiltration and inflammation leading to the proposed protective effect (FIG. 18-19). These findings indicate that Mrgprb2 molecular function is a complex system and further investigations into its precise function, its pharmacological modulation, its interaction with other molecular partners and signalling pathways would allow us to explore the receptor duality in the context of UCs.

TABLE 1
Disease activity index (DAI)

| | Stool | | % Body |
|---|---|---|---|
| Score | Consistency | Stool Blood | weight loss |
| 0 | Normal | Normal | 0-1% |
| 1 | | | 1-5% |
| 2 | Loose stools | Hemoccult positive | 5-10% |
| 3 | | | 10-15% |
| 4 | Diarrhoea | Gross bleeding | More than 15% |

TABLE 2
qRT-PCR Primer's list:

| Primer Name | Sequence (5′ to 3′) |
|---|---|
| mMrgpra1_F | GCAAGAGGAATGGGGGAAAGC |
| mMrgpra1_R | CCCGACCAGTCCGAAGATGAT |
| mMrgprb2_F | ATCAAGAATCTAAGCACCTCAGC |
| mMrgprb2_R | GAAAGCAAAATCATGGCTTGGT |
| mGapdh_F | ACTTTGTCAAGCTCATTCC |
| mGapdh_R | TGCAGCGAACTTTATTGATG |
| mOccludin_F | ACGGACCCTGACCACTATGA |
| mOccludin_R | TCAGCAGCAGCCATGTACTC |
| mEcadherin_F | GGTTTTCTACAGCATCACCG |
| mEcadherin_R | GCTTCCCCATTTGATGACAC |
| mVEGF_F | GCGGATCAAACCTCACCAAA |
| mVEGF_R | TTCACATCGGCTGTGCTGTAG |
| mClaudin 1_F | AGCTGCCTGTTCCATGTACT |
| mClaudin 1_R | CTCCCATTTGTCTGCTGCTC |

### Exemplary Products, Systems and Methods are Set Out in the Following Items:

1. A method for treating an inflammatory disorder, the method comprising administering to a subject in need thereof a composition comprising a therapeutically effective amount of compound having the structure:

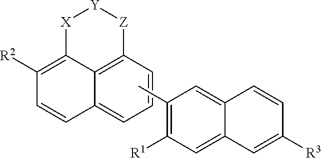

wherein each of R¹ and R³ is independently

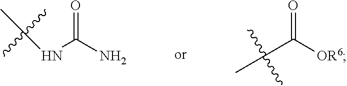

R² is H or OH;

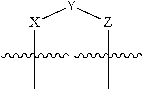

is

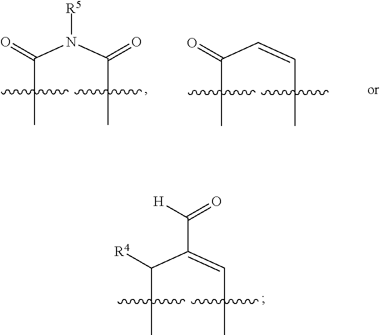

and
each of R⁴, R⁵ and R⁶ is independently H or alkyl; and a pharmaceutically acceptable carrier.
2. The method of item 1, wherein the compound is represented by the structure:

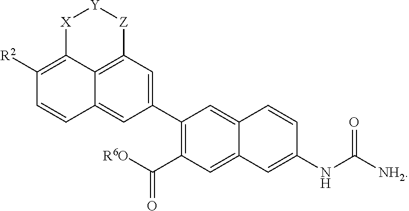

3. The method of anyone of the preceding items, wherein the compound is represented by the structure:

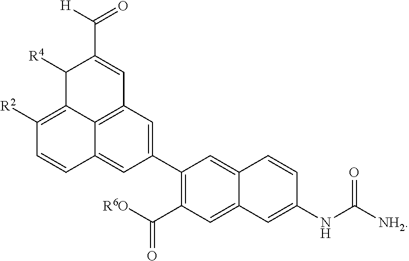

4. The method of anyone of the preceding items, wherein the compound is represented by the structure:

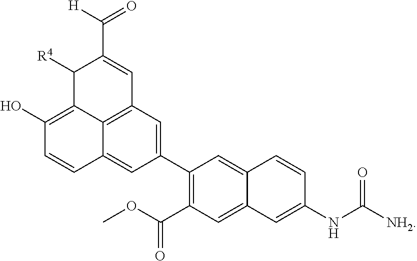

5. The method of anyone of the preceding items, wherein the compound is represented by the structure:

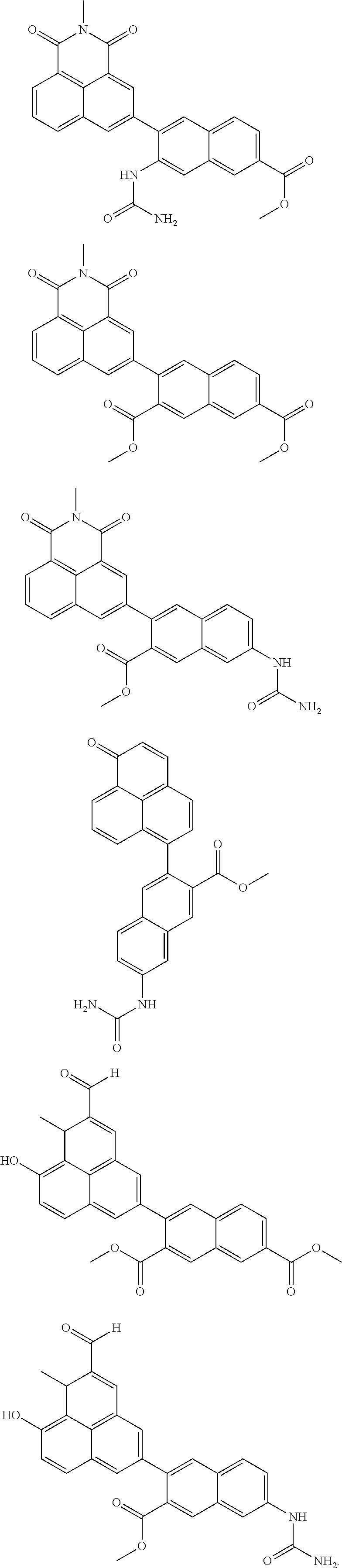

6. The method of anyone of the preceding items wherein the inflammatory disorder is an inflammation of the gastrointestinal tract.
7. The method of anyone of the preceding items wherein the inflammatory disorder is inflammatory bowel disease (“IBD”), Crohn's disease (“CD”) or ulcerative colitis (“UC”).
8. The method of anyone of the preceding items, wherein the composition is in the form of a cream, a gel, a spray, an ointment, or is a unit dosage form for oral administration.
9. The method of anyone of the preceding items, wherein the compound is present at a concentration of about 0.001 wt. % to about 10 wt. %, based on the total weight of the composition.
10. The method of anyone of the preceding items, wherein the compound is present at a concentration of about 0.1 wt. % to about 5 wt. %, based on the total weight of the composition.
11. The method of anyone of the preceding items, wherein the subject is a mammal.
12. The method of anyone of the preceding items, wherein the mammal is a human.
13. A pharmaceutical composition comprising the compound represented by the structure:

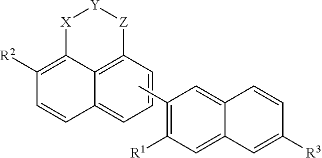

wherein each of R¹ and R³ is independently

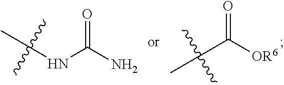

R² is H or OH;

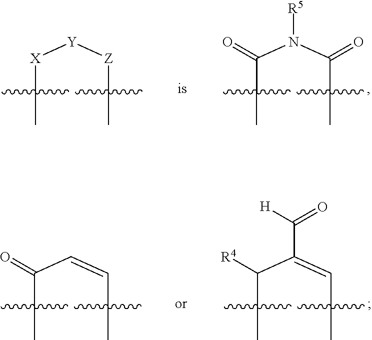

and
each of R⁴, R⁵ and R⁶ is independently H or alkyl, or a pharmaceutically acceptable salt, hydrate, solvate or isotope thereof and at least one pharmaceutically acceptable carrier.
14. The pharmaceutical composition of item 13, wherein the compound is represented by the structure:

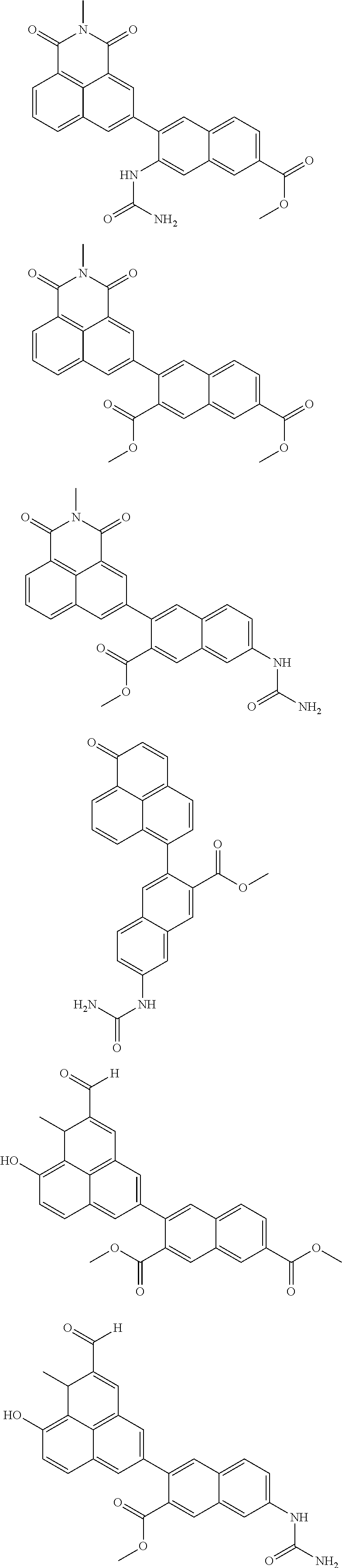

or a pharmaceutically acceptable salt, hydrate, solvate or isotope thereof and at least one pharmaceutically acceptable carrier.

The foregoing description of the specific embodiments will so fully reveal the general nature of the disclosure that others can, by applying knowledge within the skill of the relevant art(s) (including the contents of the documents cited and incorporated by reference herein), readily modify and/or adapt for various applications such specific embodiments, without undue experimentation, without departing from the general concept of the present disclosure. Such adaptations and modifications are therefore intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance presented herein, in combination with the knowledge of one skilled in the relevant art(s).

While various embodiments of the present disclosure have been described above, it should be understood that they have been presented by way of examples, and not limitation. It would be apparent to one skilled in the relevant art(s) that various changes in form and detail could be made therein without departing from the spirit and scope of the disclosure. Thus, the present disclosure should not be limited by any of the above-described exemplary embodiments but should be defined only in accordance with the following claims and their equivalents.

All references cited herein are incorporated herein by reference in their entirety and for all purposes to the same extent as if each individual publication or patent or patent application was specifically and individually indicated to be incorporated by reference in its entirety for all purposes.

### REFERENCES

1. Taku, K., et al., Ulcerative colitis (primer). Nature Reviews: Disease Primers, 2020. 6(1).
2. Cai, Z., S. Wang, and J. Li, Treatment of inflammatory bowel disease: a comprehensive review. Frontiers in medicine, 2021. 8: p. 765474.
3. do Nascimento, R.d.P., et al., Ulcerative colitis: Gut microbiota, immunopathogenesis and application of natural products in animal models. Life sciences, 2020. 258: p. 118129.
4. Vedamurthy, A. and A. N. Ananthakrishnan, Influence of environmentalfactors in the development and outcomes of inflammatory bowel disease. Gastroenterology & hepatology, 2019. 15(2): p. 72.
5. Deng, L., et al., Colonization with two different Blastocystis subtypes in DSS-induced colitis mice is associated with strikingly different microbiome and pathological features. Theranostics, 2023. 13(3): p. 1165.
6. Perše, M. and A. Cerar, Dextran sodium sulphate colitis mouse model: traps and tricks. BioMed Research International, 2012. 2012.
7. Chassaing, B., et al., Dextran sulfate sodium (DSS)-induced colitis in mice. Current protocols in immunology, 2014. 104(1): p. 15.25. 1-15.25. 14.
8. Zeng, Z., et al., Roles of G protein-coupled receptors in inflammatory bowel disease. World journal of gastroenterology, 2020. 26(12): p. 1242.
9. Ranganathan, P., et al., CXCR2 knockout mice are protected against DSS-colitis-induced acute kidney injury and inflammation. American Journal of Physiology-Renal Physiology, 2013. 305(10): p. F1422-F1427.
10. Chen, E., et al., Inflamed ulcerative colitis regions associated with MRGPRX2-mediated mast cell degranulation and cell activation modules, defining a new therapeutic target. Gastroenterology, 2021. 160(5): p. 1709-1724.
11. Wootten, D., et al., Mechanisms of signalling and biased agonism in G protein-coupled receptors. Nature reviews Molecular cell biology, 2018. 19(10): p. 638-653.
12. Hu, G.-M., T.-L. Mai, and C.-M. Chen, Visualizing the GPCR network: classification and evolution. Scientific reports, 2017. 7(1): p. 1-15.
13. Sriram, K. and P. A. Insel, G protein-coupled receptors as targets for approved drugs: how many targets and how many drugs?Molecular pharmacology, 2018. 93(4): p. 251-258.
14. Palacios, B., et al., Pharmacology of JB-9315, a new selective histamine H2-receptor antagonist. General Pharmacology: The Vascular System, 1998. 30(2): p. 181-189.
15. Kuna, P., et al., The role and choice criteria of antihistamines in allergy management—expert opinion. Advances in Dermatology and Allergology/Postȩpy Dermatologii i Alergologii, 2016. 33(6): p. 397-410.
16. Mamedova, L., et al., CysLT1 leukotriene receptor antagonists inhibit the effects of nucleotides acting at P2Y receptors. Biochemical pharmacology, 2005. 71(1-2): p. 115-125.
17. Subramanian, H., K. Gupta, and H. Ali, Roles of Mas-related G protein-coupled receptor X2 on mast cell-mediated host defense, pseudoallergic drug reactions, and chronic inflammatory diseases. Journal of Allergy and Clinical Immunology, 2016. 138(3): p. 700-710.
18. Wasserman, S. I., Mast cell biology. Journal of allergy and clinical immunology, 1990. 86(4): p. 590-593.
19. Roy, S., et al., Multifaceted MRGPRX2: New insight into the role of mast cells in health and disease. Journal of Allergy and Clinical Immunology, 2021. 148(2): p. 293-308.
20. Olivera, A., M. A. Beaven, and D. D. Metcalfe, Mast cells signal their importance in health and disease. 2018, Elsevier.
21. Kumar, M., K. Duraisamy, and B.-K.-C. Chow, Unlocking the non-IgE-mediated pseudo-allergic reaction puzzle with mas-related g-protein coupled receptor member x2 (MRGPRX2). Cells, 2021. 10(5): p. 1033.
22. Duraisamy, K., et al., P17 induces chemotaxis and differentiation of monocytes via MRGPRX2-mediated mast cell-line activation. Journal of Allergy and Clinical Immunology, 2022. 149(1): p. 275-291.
23. Green, D. P., et al., A mast-cell-specific receptor mediates neurogenic inflammation and pain. Neuron, 2019. 101(3): p. 412-420. e3.
24. Kumar, M., et al., Protective effect of genistein against compound 48/80 induced anaphylactoid shock via inhibiting MAS related G protein-coupled receptor X2 (MRGPRX2). Molecules, 2020. 25(5): p. 1028.
25. Kumar, M., et al., Novel small molecule MRGPRX2 antagonists inhibit a murine model of allergic reaction. Journal of Allergy and Clinical Immunology, 2023. 151(4): p. 1110-1122.
26. Ding, Y., et al., Quercetin inhibits Mrgprx2-induced pseudo-allergic reaction via PLCγ-IP3R related Ca2+fluctuations. International immunopharmacology, 2019. 66: p. 185-197.
27. McNeil, B. D., et al., Identification of a mast-cell-specific receptor crucial for pseudo-allergic drug reactions. Nature, 2015. 519(7542): p. 237-241.
28. Azimi, E., et al., Dual action of neurokinin-1 antagonists on Mas-related GPCRs. JCI insight, 2016. 1(16).
29. Hamilton, M. J., et al., Essential role for mast cell tryptase in acute experimental colitis. Proceedings of the National Academy of Sciences, 2011. 108(1): p. 290-295.
30. Schauber, J., et al., Heterogeneous expression of human cathelicidin hCAP18/LL-37 in inflammatory bowel diseases. European journal of gastroenterology & hepatology, 2006. 18(6): p. 615-621.
31. Shao, M., J. Liu, and H. Luo, Colitis aggravated by Mrgprb2 knockout is associated with altered immune response, intestinal barrier function and gut microbiota. Experimental Physiology, 2023. 108(1): p. 63-75.
32. Sabato, V., et al., Allergenic and mas-related G protein-coupled receptor X2-activating properties of drugs: resolving the two. The Journal of Allergy and Clinical Immunology: In Practice, 2023. 11(2): p. 395-404.
33. Xue, G., et al., Characteristics of immune cell infiltration and associated diagnostic biomarkers in ulcerative colitis: results from bioinformatics analysis. Bioengineered, 2021. 12(1): p. 252-265.
34. Schmittgen, T. D. and K. J. Livak, Analyzing real-time PCR data by the comparative CT method. Nature protocols, 2008. 3(6): p. 1101-1108.
35. Mak, S. O., L. Zhang, and B. K. Chow, In vivo actions of SCTR/AT1aR heteromer in controlling Vp expression and release via cFos/cAMP/CREB pathway in magnocellular neurons of PVN. The FASEB Journal, 2019. 33(4): p. 5389-5398.
36. Eijkelkamp, N., et al., G protein-coupled receptor kinase 6 controls chronicity and severity of dextran sodium sulphate-induced colitis in mice. Gut, 2007. 56(6): p. 847-854.
37. Benoit, C., et al., Dextran sulfate sodium (DSS)-induced colitis in mice. Curr Protoc Immunol, 2014. 25: p. 1-15.
38. Spisni, E., et al., Cyclooxygenase-2 silencing for the treatment of colitis: a combined in vivo strategy based on RNA interference and engineered Escherichia coli. Molecular Therapy, 2015. 23(2): p. 278-289.
39. Wang, D. and R. N. DuBois, The role of COX-2 in intestinal inflammation and colorectal cancer. Oncogene, 2010. 29(6): p. 781-788.
40. Occhiuto, C. J., et al., Store-operated calcium entry via STIM1 contributes to MRGPRX2 induced mast cell functions. Frontiers in immunology, 2020. 10: p. 3143.
41. Huynh, E., et al., Protective effects of protegrin in dextran sodium sulfate-induced murine colitis. Frontiers in Pharmacology, 2019. 10: p. 156.
42. Gonzalez-Rey, E., et al., Cortistatin, an antiinflammatory peptide with therapeutic action in inflammatory bowel disease. Proceedings of the National Academy of Sciences, 2006. 103(11): p. 4228-4233.
43. Dong, Y., J. Lei, and B. Zhang, Dietary quercetin alleviated DSS-induced colitis in mice through several possible pathways by transcriptome analysis. Current Pharmaceutical Biotechnology, 2020. 21(15): p. 1666-1673.
44. Zhang, Q., et al., Efficacy and Mechanism of Quercetin in the Treatment of Experimental Colitis Using Network Pharmacology Analysis. Molecules, 2022. 28(1): p. 146.
45. Benmoussa, K., et al., P17, an original host defense peptide from ant venom, promotes antifungal activities of macrophages through the induction of C-type Lectin Receptors dependent on LTB4-mediated PPARγ activation. Frontiers in immunology, 2017. 8: p. 1650.
46. Betto, T., et al., Vascular endothelial growth factor receptor 1 tyrosine kinase signaling facilitates healing of DSS-induced colitis by accumulation of Tregs in ulcer area. Biomedicine & Pharmacotherapy, 2019. 111: p. 131-141.
47. Frysz-Naglak, D., et al., Expression, localization and systemic concentration of vascular endothelial growth factor (VEGF) and its receptors in patients with ulcerative colitis. International Immunopharmacology, 2011. 11(2): p. 220-225.
48. Gao, X., et al., Change of monocytes/macrophages in ulcerative colitis patients with symptoms of anxiety and depression. BMC gastroenterology, 2023. 23(1): p. 67.
49. Klebl, F., et al., Expression of macrophage-colony stimulating factor in normal and inflammatory bowel disease intestine. The Journal of Pathology: A Journal of the Pathological Society of Great Britain and Ireland, 2001. 195(5): p. 609-615.
50. Viola, A., et al., The metabolic signature of macrophage responses. Frontiers in immunology, 2019. 10: p. 1462.
51. Kunimura, K., et al., DOCK2 regulates MRGPRX2/B2-mediated mast cell degranulation and drug-induced anaphylaxis. Journal of Allergy and Clinical Immunology, 2023. 151(6): p. 1585-1594. e9.
52. Ebach, D. R., R. Newberry, and W. F. Stenson, Differential role of tumor necrosis factor receptors in TNBS colitis. Inflammatory bowel diseases, 2005. 11(6): p. 533-540.
53. Wang, K., et al., Opposite role of tumor necrosis factor receptors in dextran sulfate sodium-induced colitis in mice. PLoS One, 2012. 7(12): p. e52924.
54. Avet, C., et al., Effector membrane translocation biosensors reveal G protein and βarrestin coupling profiles of 100 therapeutically relevant GPCRs. Elife, 2022. 11: p. e74101.
55. Gurevich, V. V. and E. V. Gurevich, GPCR signaling regulation: the role of GRKs and arrestins. Frontiers in pharmacology, 2019. 10: p. 125.
56. Wang, W., Y. Qiao, and Z. Li, New insights into modes of GPCR activation. Trends in pharmacological sciences, 2018. 39(4): p. 367-386.
57. Ng, S. Y., L. T. Lee, and B. K. Chow, Receptor oligomerization: from early evidence to current understanding in class B GPCRs. Frontiers in Endocrinology, 2013. 3: p. 175.
58. Boutin, J. A. and J. Leprince, Biased Agonism or “Biaism” for Dummies: A Commentary. Receptors, 2024. 3(1): p. 13-26.
59. Kurose, H. and S. G. Kim, Pharmacology of Antagonism of GPCR. Biological and Pharmaceutical Bulletin, 2022. 45(6): p. 669-674.
60. Bai, J., et al., Role of SCTR/AT1aR heteromer in mediating ANGII-induced aldosterone secretion. Plos one, 2019. 14(9): p. e0222005.
61. Garcia, C., et al., Deciphering biased inverse agonism of cangrelor and ticagrelor at P2Y 12 receptor. Cellular and Molecular Life Sciences, 2019. 76: p. 561-576.
62. Bellot, M., et al., Dual agonist occupancy of AT1-R-α2C-AR heterodimers results in atypical Gs-PKA signaling. Nature chemical biology, 2015. 11(4): p. 271-279.
63. Thapaliya, M. and H. Ali, GRK2 differentially regulates FceRI and MRGPRB2-mediated responses in mast cells. Frontiers in Immunology, 2023. 14: p. 1155777.
64. Rajagopal, S. and S. K. Shenoy, GPCR desensitization: Acute and prolonged phases. Cellular signalling, 2018. 41: p. 9-16.
65. Marchese, A., et al., G protein-coupled receptor sorting to endosomes and lysosomes. Annu. Rev. Pharmacol. Toxicol., 2008. 48: p. 601-629.
66. Han, S.-o., et al., MARCH2 promotes endocytosis and lysosomal sorting of carvedilol-bound β2-adrenergic receptors. Journal of Cell Biology, 2012. 199(5): p. 817-830.
67. L Mohan, M., et al., G-protein coupled receptor resensitization-appreciating the balancing act of receptor function. Current molecular pharmacology, 2012. 5(3): p. 350-361.
68. Callahan, B. N., et al., Osthole, a natural plant derivative inhibits MRGPRX2 induced mast cell responses. Frontiers in immunology, 2020. 11: p. 703.
69. Pheng, L. H., et al., Agonist-and antagonist-induced sequestration internalization of neuropeptide Y Y1 receptors in HEK293 cells. British journal of pharmacology, 2003. 139(4): p. 695-704.
70. Cawston, E. E., K. G. Harikumar, and L. J. Miller, Ligand-induced internalization of the type 1 cholecystokinin receptor independent of recognized signaling activity. American Journal of Physiology-Cell Physiology, 2012. 302(3): p. C615-C627.
71. Reubi, J. C., et al., Switch from antagonist to agonist after addition of a DOTA chelator to a somatostatin analog. European journal of nuclear medicine and molecular imaging, 2010. 37: p. 1551-1558.
72. Shiriaeva, A., et al., GPCR agonist-to-antagonist conversion: enabling the design of nucleoside functional switches for the A2A adenosine receptor. Journal of Medicinal Chemistry, 2022. 65(17): p. 11648-11657.
73. Galandrin, S., et al., Cardioprotective angiotensin-(1-7) peptide acts as a natural-biased ligand at the angiotensin II type 1 receptor. Hypertension, 2016. 68(6): p. 1365-1374.
74. Oliver, E., F. Mayor Jr, and P. D'Ocon, Beta-blockers: historical perspective and mechanisms of action. Revista Española de Cardiología (English Edition), 2019. 72(10): p. 853-862.
75. Hoffmann, C., et al., Comparative pharmacology of human β-adrenergic receptor subtypes—characterization of stably transfected receptors in CHO cells. Naunyn-Schmiedeberg's archives of pharmacology, 2004. 369: p. 151-159.
76. Khilnani, G. and A. K. Khilnani, Inverse agonism and its therapeutic significance. Indian journal of pharmacology, 2011. 43(5): p. 492.

What is claimed:

1. A method for treating an inflammatory disorder, the method comprising administering to a subject in need thereof a composition comprising a therapeutically effective amount of compound having the structure: 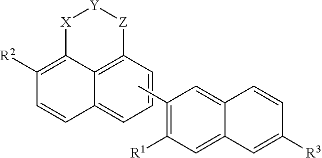
wherein each of R¹ and R³ is independently 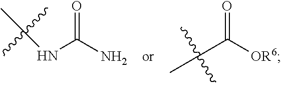
R² is H or OH; 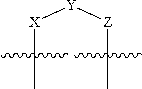
is 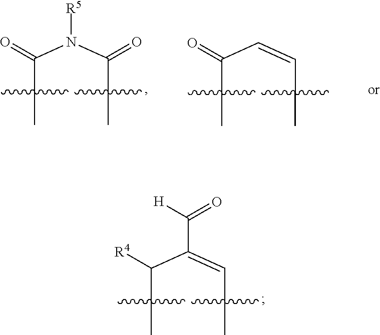
and
each of R⁴, R⁵ and R⁶ is independently H or alkyl; and a pharmaceutically acceptable carrier.

2. The method of claim 1, wherein the compound is represented by the structure:

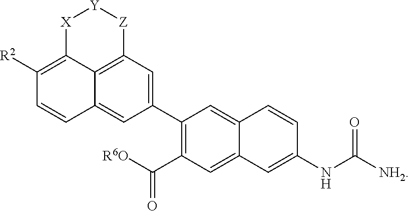

3. The method of claim 1, wherein the compound is represented by the structure:

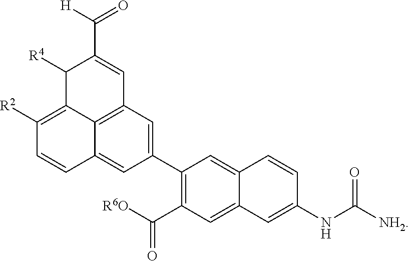

4. The method of claim 1, wherein the compound is represented by the structure:

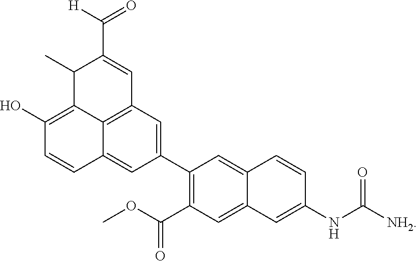 [SMILES: COC(=O)c1cc2cc(NC(N)=O)ccc2cc1-c1cc2c3c(c(O)ccc3c1)C(C)C(C=O)=C2]

5. The method of claim 1, wherein the compound is represented by the structure:

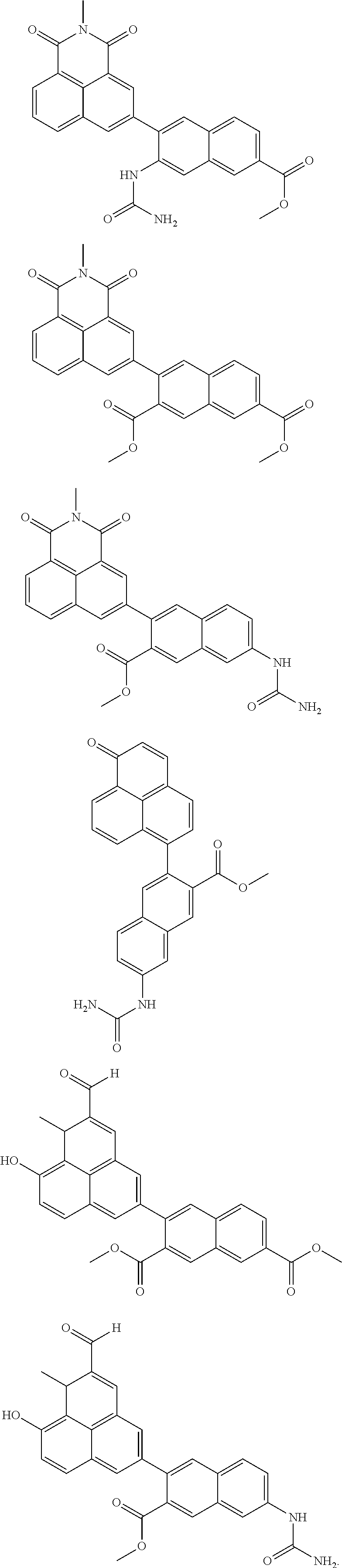

6. The method of claim 1 wherein the inflammatory disorder is an inflammation of the gastrointestinal tract.

7. The method of claim 6 wherein the inflammatory disorder is inflammatory bowel disease (“IBD”), Crohn's disease (“CD”) or ulcerative colitis (“UC”).

8. The method of claim 7, wherein the composition is in the form of a cream, a gel, a spray, an ointment, or is a unit dosage form for oral administration.

9. The method of claim 7, wherein the compound is present at a concentration of about 0.001 wt. % to about 10 wt. %, based on the total weight of the composition.

10. The method of claim 7, wherein the compound is present at a concentration of about 0.1 wt. % to about 5 wt. %, based on the total weight of the composition.

11. The method of claim 6, wherein the subject is a mammal.

12. The method of claim 11, wherein the mammal is a human.

13. A pharmaceutical composition comprising the compound represented by the structure: 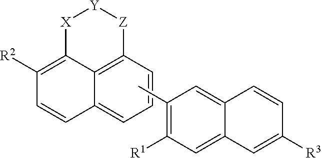
wherein each of R¹ and R³ is independently 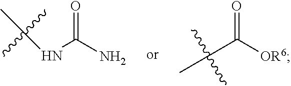
R² is H or OH; 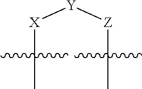
is 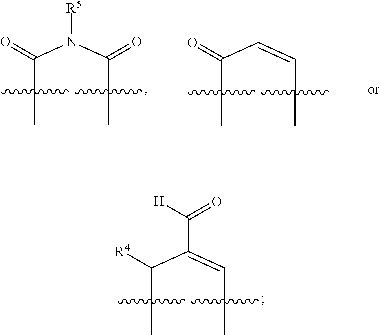
and
each of R⁴, R⁵ and R⁶ is independently H or alkyl, or a pharmaceutically acceptable salt, hydrate, solvate or isotope thereof and at least one pharmaceutically acceptable carrier.

14. The pharmaceutical composition of claim 13, wherein the compound is represented by the structure: 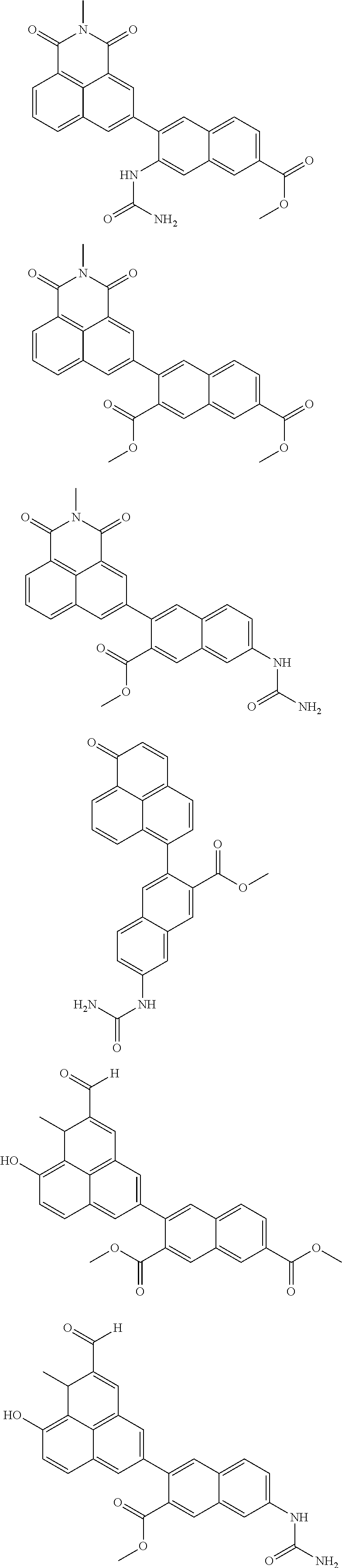
or a pharmaceutically acceptable salt, hydrate, solvate or isotope thereof and at least one pharmaceutically acceptable carrier.
